SEQUENCE LISTING (19 sequences)

SEQ ID NO: 1 - DNA, 24 bp, Artificial Sequence
  Description of Artificial Sequence Synthetic oligonucleotide
tcgtcgttttgtcgttttgtcgtt

SEQ ID NO: 2 - DNA, 20 bp, Artificial Sequence
  Description of Artificial Sequence Synthetic oligonucleotide
tccatgtcgctcctgatgct

SEQ ID NO: 3 - DNA, 20 bp, Artificial Sequence
  Description of Artificial Sequence Synthetic oligonucleotide
tccatgtcgttcctgatgct

SEQ ID NO: 4 - DNA, 24 bp, Artificial Sequence
  Description of Artificial Sequence Synthetic oligonucleotide
tcgtcgttttgtcgttttgtcgtt

SEQ ID NO: 5 - DNA, 20 bp, Artificial Sequence
  Description of Artificial Sequence Synthetic oligonucleotide
tcgtcgttgtcgttgtcgtt

SEQ ID NO: 6 - DNA, 22 bp, Artificial Sequence
  Description of Artificial Sequence Synthetic oligonucleotide
tcgtcgttgtcgttttgtcgtt

SEQ ID NO: 7 - DNA, 21 bp, Artificial Sequence
  Description of Artificial Sequence Synthetic oligonucleotide
gcgtgcgttgtcgttgtcgtt

SEQ ID NO: 8 - DNA, 21 bp, Artificial Sequence
  Description of Artificial Sequence Synthetic oligonucleotide
tgtcgtttgtcgtttgtcgtt

SEQ ID NO: 9 - DNA, 19 bp, Artificial Sequence
  Description of Artificial Sequence Synthetic oligonucleotide
tgtcgttgtcgttgtcgtt

SEQ ID NO: 10 - DNA, 14 bp, Artificial Sequence
  Description of Artificial Sequence Synthetic oligonucleotide
tcgtcgtcgtcgtt

SEQ ID NO: 11 - DNA, 20 bp, Artificial Sequence
  Description of Artificial Sequence Synthetic oligonucleotide
tcctgtcgttccttgtcgtt

SEQ ID NO: 12 - DNA, 20 bp, Artificial Sequence
  Description of Artificial Sequence Synthetic oligonucleotide
tcctgtcgttttttgtcgtt

SEQ ID NO: 13 - DNA, 21 bp, Artificial Sequence
  Description of Artificial Sequence Synthetic oligonucleotide
tcgtcgctgtctgcccttctt

SEQ ID NO: 14 - DNA, 21 bp, Artificial Sequence
  Description of Artificial Sequence Synthetic oligonucleotide
tcgtcgctgttgtcgtttctt

SEQ ID NO: 15 - DNA, 20 bp, Artificial Sequence
  Description of Artificial Sequence Synthetic oligonucleotide
tccatgacgttcctgacgtt

SEQ ID NO: 16 - DNA, 20 bp, Artificial Sequence
  Description of Artificial Sequence Synthetic oligonucleotide
gggggacgatcgtcgggggg

SEQ ID NO: 17 - DNA, 25 bp, Artificial Sequence
  Description of Artificial Sequence Synthetic oligonucleotide
tcgtcgtcgttcgaacgacgttgat

SEQ ID NO: 18 - DNA, 29 bp, Artificial Sequence
  Description of Artificial Sequence Synthetic oligonucleotide
tcgtcgttttgtcgttttgtcgttggggg

SEQ ID NO: 19 - DNA, 24 bp, Artificial Sequence
  Description of Artificial Sequence Synthetic oligonucleotide
tcgtcgttttgtcgttttgtcgtt

(19) United States
(12) Patent Application Publication
(10) Pub. No.: US 2008/0124366 A1
(43) Pub. Date: May 29, 2008

(54) Methods and Compositions for Treating Tumors

(76) Inventors: John R. Ohlfest, Coon Rapids, MN (US); Walter C. Low, Shorewood, MN (US); Wei Chen, Edina, MN (US)

Correspondence Address: VIKSNINS HARRIS & PADYS PLLP, P.O. BOX 111098, ST. PAUL, MN, 55111-1098, US

(21) Appl. No.: 11/834,483

(22) Filed: Aug. 6, 2007

Related U.S. Application Data
(60) Provisional application No. 60/821,571, filed on Aug. 6, 2006.

(51) Int. Cl. A61K 39/00; A61K 31/7052; A61K 45/00; A61K 38/21; A61K 35/74; A61K 35/76; A61P 35/04

(52) U.S. Cl. 424/278.1; 514/44; 424/85.5; 424/780; 424/93.6; 424/277.1

(57) ABSTRACT

The present invention is based on the finding that toll-like receptor (TLR) agonists affect immune responses in a subject. The compositions and methods of the present invention include administering to the subject a therapeutically effective amount of a tumor lysate or tumor lysis agent in conjunction with a therapeutically effective amount of a TLR agonist.

### RELATED APPLICATION

This patent application relates to U.S. Application Ser. No. 60/821,571 filed on Aug. 6, 2006. The instant application claims the benefit of the listed application, which is hereby incorporated by reference herein in its entirety, including the drawings.

### GOVERNMENT FUNDING

The invention described herein was made with government support under Grant Number 5 P30 CA077598 awarded by NIH/NCI. The United States Government has certain rights in the invention.

### FIELD OF THE INVENTION

This invention relates generally and specifically to the use of toll-like receptor (TLR) agonists in conjunction with tumor lysates and/or tumor lysis agents to treat tumors.

### BACKGROUND OF THE INVENTION

Toll-like receptors (TLRs) are type I transmembrane proteins that recognize pathogens and activate immune cell responses as a key part of the innate immune system. In vertebrates, they can help activate the adaptive immune system, linking innate and acquired immune responses. TLRs are pattern recognition receptors (PRRs), binding to pathogen-associated molecular patterns, small molecular sequences consistently found on pathogens.

It has been estimated that most mammalian species have between ten and fifteen types of Toll-like receptors. Eleven TLRs (named simply TLR1 to TLR11) have been identified in humans, and equivalent forms of many of these have been found in other mammalian species. TLRs function as a dimer. Though most TLRs appear to function as homodimers, TLR2 forms heterodimers with TLR1 or TLR6, each dimer having different ligand specificity. The function of TLRs in all organisms appears to be similar enough to use a single model of action. Each Toll-like receptor forms either a homodimer or heterodimer in the recognition of a specific or set of specific molecular determinants present on microorganisms.

Because the specificity of Toll-like receptors (and other innate immune receptors) cannot be changed, these receptors must recognize patterns that are constantly present on threats, not subject to mutation, and highly specific to threats (i.e., not normally found in the host where the TLR is present). Patterns that meet this requirement are usually critical to the pathogen's function and cannot be eliminated or changed through mutation; they are said to be evolutionarily conserved. Well conserved features in pathogens include bacterial cell-surface lipopolysaccharides (LPS), lipoproteins, lipopeptides and lipoarabinomannan; proteins such as fagellin from bacterial flagella; double-stranded RNA of viruses or the unmethylated CpG islands of bacterial and viral DNA; and certain other RNA and DNA.

Bacterial DNA, but not vertebrate DNA, has direct immunostimulatory effects on peripheral blood mononuclear cells (PBMC) in vitro (Krieg, A. M. et al., Nature 374: 546-549 (1995)). This lymphocyte activation is due to unmethylated CpG dinucleotides, which are present at the expected frequency in bacterial DNA ( 1/16), but are under-represented (CpG suppression, 1/50 to 1/60) and methylated in vertebrate DNA. Activation may also be triggered by addition of synthetic oligodeoxynucleotides (ODN) that contain an unmethylated CpG dinucleotide in a particular sequence context. It appears likely that the rapid immune activation in response to CpG DNA may have evolved as one component of the innate immune defense mechanisms that recognize structural patterns specific to microbial molecules.

CpG DNA induces proliferation of almost all (>95%) B cells and increases immunoglobulin (Ig) secretion. This B cell activation by CpG DNA is T cell independent and antigen non-specific. However, B cell activation by low concentrations of CpG DNA has strong synergy with signals delivered through the B cell antigen receptor for both B cell proliferation and Ig secretion (Krieg, A. M. et al., Nature 374: 546-549 (1995)). This strong synergy between the B cell signaling pathways triggered through the B cell antigen receptor and by CpG DNA promotes antigen specific immune responses. In addition to its direct effects on B cells, CpG DNA also directly activates monocytes, macrophages, and dendritic cells to secrete a variety of cytokines, including high levels of IL-12. These cytokines stimulate natural killer (NK) cells to secrete g-interferon (IFN-gamma) and have increased lytic activity. Overall, CpG DNA induces a T_{H}1 like pattern of cytokine production dominated by IL-12 and IFN-gamma with little secretion of T_{H}2 cytokines.

There is a need for new, effective compositions and treatments for tumors.

### SUMMARY OF THE INVENTION

The present inventors have developed a dendritic cell-free pharmaceutical composition that contains a toll-like receptor (TLR) agonist and a tumor lysate and/or tumor lysis agent in a pharmaceutically acceptable carrier. This pharmaceutical composition is administered in vivo via any number of routes such as intramuscular, subcutaneous, or into a tumor in a subject. The present method does not involve the extraction of dendritic cells from the subject prior to the administration of the pharmaceutical composition. Thus, the present composition is a significantly simplified improvement over procedures in used by others.

The present invention provides a pharmaceutical composition that includes a toll-like receptor (TLR) agonist and a tumor lysate and/or a tumor lysis agent in a pharmaceutically acceptable carrier. In certain embodiments, the TLR agonist is a TLR-9 agonist or a TLR-3 agonist. In certain embodiments, the TLR agonist is an oligonucleotide containing an immunostimulatory CpG motif. In certain embodiments, the pharmaceutical composition contains more than one type of oligonucleotide. The oligonucleotide may be from about 8 to about 1000 bases in length (or any integer in between), such as from about 8 to about 30 bases in length. The oligonucleotide may have a natural phosphodiester backbone, a completely or partially a synthetic backbone, or a completely synthetic phosphorothioate backbone. In certain embodiments, the oligonucleotide is made with a chimeric backbone with synthetic phosphorothioate linkages at the 3′ and 5′ ends and natural phosphodiester linkages in the CpG-containing center to form a chimeric oligonucleotide. In certain embodiments, the oligonucleotide is made with a chimeric backbone that is made with synthetic phosphorothioate linkages for five linkages at the 3′ end and two linkages at the 5′ end, and with natural phosphodiester linkages in between.

In certain embodiments, the pharmaceutical composition contains an oligonucleotide that has a formula 5′-N₁X₁CGX₂N₂-3′, wherein at least one nucleotide separates consecutive CpGs; X₁ is adenine, guanine or thymidine; X₂ is cytosine, adenine, or thymine; N₁ is a nucleic acid of about 0-26 bases, and N₂ is a nucleic acid of about 0-26 bases. In certain embodiments, the oligonucleotide is from 8 to about 1000 bases in length. In certain embodiments, neither N₁ nor N₂ contains a CCGG quadmer or more than one CGG trimer, and the oligonucleotide is from about 8-30 bases in length.

In certain embodiments, the pharmaceutical composition contains an oligonucleotide that has a formula 5′-N₁X₁X₂CGX₃X₄N₂-3′, wherein at least one nucleotide separates consecutive CpGs; X₁X₂ is selected from the group consisting of TpT, CpT, TpC, ApT, GpT, GpG, GpA, and ApA; X₃X₄ is selected from the group consisting of GpT, GpA, ApA, ApT, TpT and CpT; N₁ is a nucleic acid of about 0-26 bases, and N₂ is a nucleic acid of about 0-26 bases. In certain embodiments, N₁ and N₂ do not contain a CCGG quadmer or more than one CGG trimer and the oligonucleotide is from about 8-1000 bases in length (or any integer in-between). In certain embodiments the oligonucleotide is from about 8-30 bases in length.

In certain embodiments, the oligonucleotide is ODN 1826 (mouse) 5′-TCCATGACGTTCCTGACGTT-3′ (SEQ ID NO:15), ODN 2216 (type A, human) 5′-GGGGGACGATCGTCGGGGGG-3′ (SEQ ID NO:16), ODN M362 (type C, human) 5′-TCGTCGTCGTTCGAACGACGTTGAT-3′ (SEQ ID NO:17).

In one embodiment, the oligonucleotide is 5′-TCGTCGTTTTGTCGTTTTGTCGTT-3′ (SEQ ID NO:1; OND PF-3512676, also called ODN 2006 or 7909).

In one embodiment, the oligonucleotide is the ODN 2006-G5 sequence 5′-TCGTCGTTTTGTCGTTTTGTCGTTGGGGG-3′ (SEQ ID NO:18).

In certain embodiments, the oligonucleotide is one or more of the following:

| | |
|---|---|
| 5′-TCCATGTCGCTCCTGATGCT-3′; | (SEQ ID NO:2) |
| 5′-TCCATGTCGTTCCTGATGCT-3′; | (SEQ ID NO:3) |
| 5′-TCGTCGTTTTGTCGTTTTGTCGTT-3′; | (SEQ ID NO:4) |
| 5′-TCGTCGTTGTCGTTGTCGTT-3′; | (SEQ ID NO:5) |
| 5′-TCGTCGTTGTCGTTTTGTCGTT-3′; | (SEQ ID NO:6) |
| 5′-GCGTGCGTTGTCGTTGTCGTT-3′; | (SEQ ID NO:7) |
| 5′-TGTCGTTTGTCGTTTGTCGTT-3′; | (SEQ ID NO:8) |
| 5′-TGTCGTTGTCGTTGTCGTT-3′; | (SEQ ID NO:9) |
| 5′-TCGTCGTCGTCGTT-3′; | (SEQ ID NO:10) |
| 5′-TCCTGTCGTTCCTTGTCGTT-3′; | (SEQ ID NO:11) |
| 5′-TCCTGTCGTTTTTTGTCGTT-3′; | (SEQ ID NO:12) |
| 5′-TCGTCGCTGTCTGCCCTTCTT-3′; | (SEQ ID NO:13) |
| 5′-TCGTCGCTGTTGTCGTTTCTT-3′; | (SEQ ID NO:14) |
| 5′-TCCATGACGTTCCTGACGTT-3′; | (SEQ ID NO:15) |
| 5′-GGGGGACGATCGTCGGGGGG-3′; | (SEQ ID NO:16) |
| 5′-TCGTCGTCGTTCGAACGACGTTGAT-3′; | (SEQ ID NO:17) |
| and | |
| 5′-TCGTCGTTTTGTCGTTTTGTCGTTGGGGG-3′. | (SEQ ID NO:18) |

In certain embodiments, the TLR-3 agonist is poly-ICLC.

In certain embodiments, the pharmaceutical composition further includes at least one additional adjuvant, such as aluminum (alum). For example, the adjuvant may be aluminum hydroxide. In one embodiment, incomplete Freud's adjuvant may be used.

In certain embodiments, the pharmaceutical composition further includes interferon-gamma (INF-gamma).

In certain embodiments, the pharmaceutical composition further includes a vector encoding interferon-gamma. In certain embodiments, the vector is a plasmid vector. In certain embodiments, the vector is a viral vector. In certain embodiments, the vector is plasmid DNA. In certain embodiments, the vector is an RNA vector. In certain embodiments, the vector is a transposon-based plasmid (e.g., Sleeping Beauty, Tol 2, Frog Prince), or an integrase-based plasmid (e.g., a C31 phage integrease). In certain embodiments, the vector is an integrating plasmid. In certain embodiments, the vector is an episomal plasmid. In one embodiment, the plasmid is a Sleeping Beauty-based plasmid. Examples of some viral vectors include lentiviral, retroviral, adenoviral, adeno-associated viral, herpes virus, chimeric viruses, and oncoloytic viral.

In certain embodiments, the TLR agonist is a combination of more than one type of oligonucleotide.

In certain embodiments, the tumor lysis agent is a chemotherapy drug or biological toxin. In certain embodiments, the tumor lysis agent is diphtheria toxin, temozolomide (Temodar®), Temodar, Carboplatin, Doxyrubicin, or a replication competent CMV virus.

The present invention also provides methods of inducing a therapeutic immune response in a subject having or at risk of having a tumor by administering to the subject a therapeutically effective amount of a pharmaceutical composition described herein above.

The present invention also provides methods of preventing metastatic spread of a tumor in a subject having received a primary therapy comprising administering the pharmaceutical composition described herein above.

The present invention provides further provides methods of inducing an immune response in a subject, comprising administering to the subject a therapeutically effective amount of a pharmaceutical composition described herein above.

The present invention also provides methods of inducing a therapeutic immune response in a subject having or at risk of having a tumor by administering to the subject a tumor lysate and a toll-like receptor (TLR) agonist.

The present invention provides further provides methods of inducing an immune response in a subject, comprising administering to the subject a therapeutically effective amount of a tumor lysate and a toll-like receptor (TLR) agonist.

In certain embodiments of the present methods, the TLR agonist is a TLR-9 agonist or a TLR-3 agonist. In certain embodiments, the tumor lysate and/or tumor lysis agent and the TLR agonist are administered simultaneously. In certain embodiments, the tumor lysate and/or tumor lysis agent and the TLR agonist are mixed ex vivo. In certain embodiments, the tumor lysate and/or tumor lysis agent and the TLR agonist are administered separately within 21 days of each other (or any time period between 0 and 21 days), such as within 2-5 days of each other. In certain embodiments, the tumor lysis agent and/or tumor lysate and the TLR agonist are administered multiple times, such as 2-5 times.

In the methods of the present invention, the subject may be a vertebrate animal including a human, dog, cat, horse, cow, pig, sheep, goat, chicken, monkey, rat, or mouse. In certain embodiments, the tumor lysate contains lysed tumor cells from the subject. In certain embodiments, the tumor lysate is generated from an allogenic cell line. In certain embodiments, the tumor lysis agent and/or tumor lysate and the TLR agonist are mixed ex vivo.

In certain embodiments, the pharmaceutical composition is administered intratumorally. In certain embodiments, the method further involves administering interferon-gamma or a vector encoding interferon-gamma. In certain embodiments the interferon-gamma or a vector encoding interferon-gamma is administered intratumorally.

In addition to the treatment of active disorders, the methods and compositions of the invention are used prophylactically or after tumor diagnosis.

### BRIEF DESCRIPTION OF THE DRAWINGS

FIG. 1. This Figure depicts results demonstrating that INF-γ gene transfer increased survival.

FIG. 2: This Figure depicts results demonstrating that the administration of tumor lysate and CpG ODN(s) is an effective therapy for cancer.

FIG. 3A: This Figure depicts results demonstrating that the combination of INF-γ gene transfer and the administration of tumor lysate and CpG ODN(s) is an effective treatment for cancer. FIG. 3A depicts results from mice treated with “All Combined Therapy” (CpG+tumor lysates combined with IFN-gamma) (4 weeks). FIG. 3B depicts results from mice treated with saline (4 weeks).

FIG. 4: This Figure depicts an example of an experimental design for INF-γ and CpG+tumor lysate treatment.

FIGS. 5A-D. CpG/lysate vaccination is associated with accumulation of T cells in the cervical lymph nodes. C57B1/6 mice were vaccinated with CpG/lysate, lysate, CpG, or untreated “normal” and the cervical lymph nodes were analyzed by flow cytometry. (A) The total cells recovered from each lymph node are shown. The absolute number of CD3⁺ (B), CD3⁺ CD4⁺ (C), and CD3⁺ CD8⁺ (D) cells are shown. The error bars indicate standard deviation (* p<0.05 vs. lysate or normal, ** p<0.05 vs. CpG).

FIGS. 6A-C. CpG/lysate vaccination caused accumulation of activated DCs in the draining lymph nodes and generated tumor-reactive lymphocytes. Cervical lymph nodes were collected from mice treated identically to FIG. 1 and analyzed by flow cytometry. (A) Dendritic cells were analyzed for expression of activation markers CD86 and CCR7. Each data point represents the absolute number of DCs per lymph node harvested (* p<0.05 vs. lysate or normal, ** p<0.05 vs. CpG). (B) Tumor-bearing mice were vaccinated with CpG/lysate, or lysate, saline, CpG alone. Splenocytes were harvested to determine IFN-gamma elaboration in response to GL261 and GL261-Luc, or C6 as an irrelevant control in the ELISPOT assay. Error bars represent standard deviation (* indicates p<0.001 comparing to saline, CpG or lysate group). (C) Tumor-bearing mice were vaccinated with CpG/lysate, lysate or saline. Splenocytes were harvested and incubated with GL261 and GL261-Luc, or C6 as an irrelevant control to determine their cytotoxic activity in the CTL assay. The results from one representative animal in each group are shown.

FIGS. 7A-B. Vaccination inhibited tumor growth and significantly extended survival. (A) Glioma bearing mice were vaccinated with CpG/lysate, or lysate, saline, or CpG alone. Bioluminescent tumor imaging conducted following tumor implantation is plotted as measured photons/second/cm². Each line represents the measured photons from a single mouse over time (black lines are saline-treated, red lines are CpG/lysate-treated). (B) Cumulative survival of mice from A. Log rank statistical analysis from mice vaccinated with CpG plus parental GL261 lysate (WT) or GL261-Luc lysate survived significantly longer than all other groups (p<0.001).

### DETAILED DESCRIPTION OF EMBODIMENTS

It is to be understood that this invention is not limited to the particular methodology, protocols, sequences, models and reagents described as such may, of course, vary. It is also to be understood that the terminology used herein is for the purpose of describing particular embodiments only, and is not intended to limit the scope of the present invention, which will be limited only by the appended claims.

All publications mentioned herein are incorporated herein by reference for the purpose of describing and disclosing the oligonucleotides and methodologies that are described in the publications that might be used in connection with the presently described invention.

As used herein the article “a” or “an” is used to mean “one or more.” For example “an oligonucleotide” would mean “one or more oligonucleotide.”

Oligonucleotides

Toll-like receptor 9 (TLR9) recognizes unmethylated bacterial CpG DNA and initiates a signaling cascade leading to the production of proinflammatory cytokines. The stimulatory effect of CpG DNA is conferred by unmethylated CpG dinucleotides in particular base contexts (CpG motifs) that also determine the species-specific activity of CpG DNA. CpG motifs containing the core sequence GACGTT highly stimulate mouse TLR9, whereas CpG motifs containing more than one CpG and the core sequence GTCGTT are optimal inducers of human TLR9.

Accumulating evidence suggests that CpG DNA and TLR9 interact in intracellular compartments. For example, lipofection increases the stimulatory activity of CpG DNA and chloroquine, an inhibitor of endosomal acidification, prevents TLR9 signaling.

Recent studies show that TLR9 is expressed in the ER of resting cells in contrast to most TLRs that are located on the plasma membrane (Latz E. et al., 2004. Nat. Immunol. 5(2):190-8). As CpG DNA is internalized through endocytosis, TLR9 relocates to the entry site of CpG DNA. The accumulation of CpG DNA and TLR9 in the endosomes leads to their co-localization within the same vesicles, and induces the recruitment of MyD88 to initiate signaling (Takeshita F. et al., 2004. Semin Immunol. 16(1):17-22).

CpG DNA binds directly to TLR9. A potential CpG-DNA binding domain was identified within TLR9 that shares homology with the methyl-CpG-DNA binding domain (MBD) of MBD proteins, a family of proteins implicated in gene silencing and chromatin remodelling (Rutz M. et al., 2004. Eur J. Immunol. 34(9):2541-50).

TLR9 recognizes specifically CpG DNA that is unmethylated and single stranded (ss). Methylation of the cytosine within the CpG motif strongly reduces the affinity of TLR9 (Rutz M. et al., 2004. Eur J. Immunol. 34(9):2541-50; Cornelie S. et al., 2004. J Biol Chem. 279(15):15124-9). In addition, double stranded (ds) CpG DNA is a weak stimulator of TLR9 compared to its ss counterpart (Rutz M. et al., 2004. Eur J Immunol. 34(9):2541-50). This observation seems to contradict the findings that genomic E. coli DNA activates TLR9. Others have found that E. coli DNA induces a poor response in TLR9-transfected HEK293 cells.

In contrast, some researchers have observed that short ss fragments of E. coli DNA, generated by sonication and denaturation, were able to activate TLR9. A possible explanation is that upon endocytosis, ds CpG DNA is degraded into small ss CpG motifs that can activate TLR9.

CpG DNA containing a phosphodiester (PD) backbone interacts with TLR9 in a CpG sequence specific manner. In contrast, phosphorothioate (PTO)-protected ODNs bind to TLR9 in a CpG-independent manner (Takeshita F. et al., 2004. Semin Immunol. 16(1):17-22; Rutz M. et al., 2004. Eur J Immunol. 34(9):2541-50), but show a CpG-dependent stimulatory activity. This difference between PD and PTO backbones suggests that the structure of the ODN influences the binding to TLR9 and the subsequent cellular activation.

Three major classes of CpG ODN that are structurally and phenotypically distinct have been described. Examples of each class are shown in Krieg (Krieg, 2006, Nature Reviews Drug Discovery, 5, 471-484) together with the immune effects and structural characteristics that are specific to the class. The A-class CpG ODN (also referred to as type D) are potent inducers of interferon-α (IFNα) secretion (from plasmacytoid dendritic cells), but only weakly stimulate B cells. The structures of A-class ODN include poly-G motifs (three or more consecutive guanines) at the 5′ and/or 3′ ends that are capable of forming very stable but complex higher-ordered structures known as G-tetrads, and a central phosphodiester region containing one or more CpG motifs in a self-complementary palindrome. These motifs cause A-class ODN to self-assemble into nanoparticles. B-class ODN (also referred to as type K) have a phosphorothioate backbone, do not typically form higher-ordered structures, and are strong B-cell stimulators but weaker inducers of IFNα secretion. However, if B-class CpG ODN are artificially forced into higher-ordered structures on beads or microparticles, in dendrimers or with cationic lipid transfection, they exert the same immune profile as the A-class CpG ODN, thereby linking the formation of higher-ordered structures to biological activity. The C-class CpG ODN have immune properties intermediate between the A and B classes, inducing both B-cell activation and IFNα secretion. These properties seem to result from the unique structure of these ODN, with one or more 5′ CpG motifs, and a 3′ palindrome, which is thought to allow duplex formation within the endosomal environment (Krieg, 2006. Nature Reviews Drug Discovery, 5, 471-484; Takeshita F. et al., 2004. Semin Immunol. 16(1):17-22; Verthelyi D, Zeuner R A., 2003. Trends Immunol. 24:519-522).

CpG ODNs are synthetic oligonucleotides that contain unmethylated CpG dinucleotides in particular sequence contexts (CpG motifs). CpG motifs containing the core sequence GACGTT highly stimulate mouse TLR9, whereas CpG motifs containing more than one CpG and the core sequence GTCGTT are optimal inducers of human TLR9.

These CpG motifs are present at a 20-fold greater frequency in bacterial DNA compared to mammalian DNA. They induce a coordinated set of immune responses based on the activation of immune cells primarily involved in the recognition of these molecules. Two types of CpG ODNs have been identified based on their distinct activity on plasmacytoid dendritic cells (PDC), key sensors of the CpG motifs (Krug A. et al., 2001. Eur J Immunol, 31(7): 2154-63). CpG-A is a potent inducer of IFN-α in plasmacytoid dendritic cells (PDC), whereas CpG-B is a weak inducer of IFN-α but a potent activator of B cells. Although the CpG motifs differ between mice and humans, in both species the recognition of CpG ODNs is mediated primarily by TLR9 (Bauer S. et al., 2001. Proc Natl Acad Sci USA, 98(16):9237-42).

A new type of CpG ODN has been recently identified, termed CpG-C, with both high induction of PDC and activation of B cells (Hartmann G. et al., 2003. Eur J Immunol. 33(6):1633-41). The sequence of CpG-C combines elements of both CpG-A and CpG-B. The most potent sequence is called M362, which contains a central palindromic sequence with CG dinucleotides, a characteristic feature of CpG-A, and a “TCGTCG motif” at the 5′ end, present in CpG-B.

Others have used oligodeoxynucleotides containing CpG motifs (CpG ODNs) to display a strong immunostimulating activity and drive the immune response toward the Th1 (T helper type 1) phenotype. These ODNs showed promising efficacy in preclinical studies when injected locally in several cancer models. A phase 1 trial was conducted to define the safety profile of CpG-28, a phosphorothioate CpG ODN, administered intratumorally by convection-enhanced delivery in patients with recurrent glioblastoma. Cohorts of three to six patients were treated with escalating doses of CpG-28 (0.5-20 mg), and patients were observed for at least four months. Twenty-four patients entered the trial. All patients had previously been treated with radiotherapy, and most patients had received one or several types of chemotherapy. Median age was 58 years (range, 25-73) and median KPS was 80% (range, 60%-100%). Adverse effects possibly or probably related to the studied drug were moderate and consisted mainly in worsening of neurological conditions (four patients), fever above 38° C. that disappeared within a few days (five patients), and reversible grade 3 lymphopenia (seven patients). Only one patient experienced a dose-limiting toxicity. Preliminary evidence of activity was suggested by a minor response observed in two patients and an overall median survival of 7.2 months. In conclusion, CpG-28 was well tolerated at doses up to 20 mg per injection in patients with recurrent glioblastoma. The main side effects were limited to transient worsening of neurological condition and fever.

Thus, previous scientists injected CpG ODNs directly into gliomas. Unfortunately, in clinical trials, most of the patients still died. This procedure causes seizures when injected into the brain, which is what half of the patients in this trial experienced.

Oligonucleotides

The term “nucleic acid” or “oligonucleotide” refers to a polymeric form of nucleotides at least five bases in length. The term “oligonucleotide” includes both single and double-stranded forms of nucleic acid. The nucleotides of the invention can be deoxyribonucleotides, ribonucleotides, or modified forms of either nucleotide. Generally, double-stranded molecules are more stable in vivo, although single-stranded molecules have increased activity when they contain a synthetic backbone.

An “oligodeoxyribonucleotide” (ODN) as used herein is a deoxyribonucleic acid sequence from about 3-1000 (or any integer in between) bases in length. In certain embodiments, the ODN is about 3 to about 50 bases in length. Lymphocyte ODN uptake is regulated by cell activation. For example, B-cells that take up CpG ODNs proliferate and secrete increased amounts of immunoglobulin. The present invention is based on the finding that certain oligonucleotides containing at least one unmethylated cytosine-guanine (CpG) dinucleotide activate the immune response.

A “CpG” or “CpG motif” refers to a nucleic acid having a cytosine followed by a guanine linked by a phosphate bond. The term “methylated CpG” refers to the methylation of the cytosine on the pyrimidine ring, usually occurring at the 5-position of the pyrimidine ring. The term “unmethylated CpG” refers to the absence of methylation of the cytosine on the pyrimidine ring. Methylation, partial removal, or removal of an unmethylated CpG motif in an oligonucleotide of the invention is believed to reduce its effect. Methylation or removal of all unmethylated CpG motifs in an oligonucleotide substantially reduces its effect. The effect of methylation or removal of a CpG motif is “substantial” if the effect is similar to that of an oligonucleotide that does not contain a CpG motif.

In certain embodiments the CpG oligonucleotide is in the range of about 8 to 1000 bases in size, or about 8 to 30 bases in size. For use in the present invention, the nucleic acids can be synthesized de novo using any of a number of procedures well known in the art. For example, the cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986; Garegg et al., Tet. Let. 27:4055-4058, 1986, Gaffney et al., Tet. Let. 29:2619-2622, 1988). These chemistries can be performed by a variety of automated oligonucleotide synthesizers available in the market.

Alternatively, CpG dinucleotides can be produced on a large scale in plasmids, (see Sambrook, T., et al., Molecular Cloning: A Laboratory Manual, Cold Spring Harbor laboratory Press, New York, 1989), which after being administered to a subject, are degraded into oligonucleotides. Oligonucleotides can be prepared from existing nucleic acid sequences (e.g., genomic or cDNA) using known techniques, such as those employing restriction enzymes, exonucleases or endonucleases.

The CpG oligonucleotides of the invention are immunostimulatory molecules. An “immunostimulatory nucleic acid molecule” refers to a nucleic acid molecule, which contains an unmethylated cytosine, guanine dinucleotide sequence (i.e., “CpG DNA” or DNA containing a cytosine followed by guanosine and linked by a phosphate bond) and stimulates (e.g., has a mitogenic effect on, or induces or increases cytokine expression by) a dendritic cell. An immunostimulatory nucleic acid molecule can be double-stranded or single-stranded. Generally, double-stranded molecules are more stable in vivo, while single-stranded molecules have increased immune activity.

A “nucleic acid” or “DNA” means multiple nucleotides (i.e., molecules comprising a sugar (e.g., ribose or deoxyribose) linked to a phosphate group and to an exchangeable organic base, which is either a substituted pyrimidine (e.g., cytosine (C), thymine (T) or uracil (U)) or a substituted purine (e.g., adenine (A) or guanine (G)). As used herein, the term refers to ribonucleotides as well as oligodeoxyribonucleotides. The term shall also include polynucleosides (i.e., a polynucleotide minus the phosphate) and any other organic base containing polymer. Nucleic acid molecules can be obtained from existing nucleic acid sources (e.g., genomic or cDNA), but are preferably synthetic (e.g., produced by oligonucleotide synthesis).

In one embodiment, the nucleic acid sequences useful in the methods of the invention are represented by the formula:

5′-N₁X₁CGX₂N₂-3′

wherein at least one nucleotide separates consecutive CpGs; X₁ is adenine, guanine or thymidine; X₂ is cytosine, adenine, or thymine; and each of N₁ and N₂ is from about 0-26 bases. In certain embodiments, neither N₁ nor N₂ contain a CCGG quadmer or more than one CGG trimer. In certain embodiments, the oligonucleotide is from about 8-30 bases in length. However, nucleic acids of any size (even many kb long) can be used in the invention if CpGs are present, as larger nucleic acids are degraded into oligonucleotides inside cells. Such synthetic oligonucleotides do not include a CCGG quadmer or more than one CCG or CGG trimer at or near the 5′ or 3′ terminals and/or the consensus mitogenic CpG motif is not a palindrome.

In another embodiment, the method of the invention includes the use of an oligonucleotide that contains a CpG motif represented by the formula:

5′-N₁X₁X₂CGX₃X₄N₂-3′

wherein at least one nucleotide separates consecutive CpGs; X₁X₂ is selected from the group consisting of TpT, CpT, TpC, ApT, GpT, GpG, GpA, and ApA; X₃X₄ is selected from the group consisting of GpT, GpA, ApA, ApT, TpT and CpT; and each of N₁ and N₂ is from about 0-26 bases. In certain embodiments, neither N₁ nor N₂ contain a CCGG quadmer or more than one CCG or CGG trimer. In certain embodiments, the oligonucleotide is from about 8-1000 bases in length. In certain embodiments the oligonucleotide is from about 8-30 bases in length, but may be of any size (even many kb long) if sufficient immunostimulatory motifs are present, since such larger nucleic acids are degraded into smaller oligonucleotides inside of cells. Synthetic oligonucleotides of this formula do not include a CCGG quadmer or more than one CCG or CGG trimer at or near the 5′ and/or 3′ terminals and/or the consensus mitogenic CpG motif is not a palindrome. Other CpG oligonucleotides can be assayed for efficacy using methods described herein. In one embodiment, the oligonucleotide comprises the sequence 5′-TCGTCGTTTTGTCGTTTTGTCGTT-3′ (SEQ ID NO:1).

In certain embodiments, the immunostimulatory nucleic acid sequences of the invention include X₁X₂ selected from the group consisting of GpT, GpG, GpA and ApA and X₃X₄ is selected from the group consisting of TpT, CpT and GpT. In certain embodiments, for facilitating uptake into cells, CpG containing immunostimulatory nucleic acid molecules are in the range of 8 to 30 bases in length.

A prolonged effect can be obtained using stabilized oligonucleotides, where the oligonucleotide incorporates a phosphate backbone modification (e.g., a phosphorothioate or phosphorodithioate modification). For example, the phosphate backbone modification occurs at the 5′ end of the nucleic acid for example, at the first two nucleotides of the 5′ end of the nucleic acid. Further, the phosphate backbone modification may occur at the 3′ end of the nucleic acid for example, at the last five nucleotides of the 3′ end of the nucleic acid. Preferred nucleic acids containing an unmethylated CpG have a relatively high stimulation with regard to B cell, monocyte, and/or natural killer cell responses (e.g., induction of cytokines, proliferative responses, lytic responses, among others).

For use in vivo, nucleic acids are preferably relatively resistant to degradation (e.g., via endo- and exo-nucleases). Secondary structures, such as stem loops, can stabilize nucleic acids against degradation. Alternatively, nucleic acid stabilization can be accomplished via phosphate backbone modifications. In certain embodiments, a stabilized nucleic acid that has at least a partial phosphorothioate modified backbone is used. Phosphorothioates may be synthesized using automated techniques employing either phosphoramidate or H-phosphonate chemistries. Aryl- and alkyl-phosphonates can be made, e.g., as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., Chem. Rev. 90: 544, 1990; Goodchild, J., Bioconjugate Chem. 1: 165, 1990).

In certain embodiments, the immunostimulatory CpG DNA is in the range of between 8 to 30 bases in size when it is an oligonucleotide. Alternatively, CpG dinucleotides can be produced on a large scale in plasmids, which after being administered to a subject are degraded into oligonucleotides. Preferred immunostimulatory nucleic acid molecules (e.g., for use in increasing the effectiveness of a vaccine or to treat an immune system deficiency by stimulating an antibody (i.e., humoral response in a subject) have a relatively high stimulation index with regard to B cell, dendritic cell and/or natural killer cell responses (e.g., cytokine, proliferative, lytic or other responses).

As used herein the term “palindromic sequence” means an inverted repeat (i.e., a sequence such as ABCDEE′D′C′B′A′ in which A and A′ are bases capable of forming the usual Watson-Crick base pairs. In vivo, such sequences may form double-stranded structures.

A “stabilized nucleic acid molecule” shall mean a nucleic acid molecule that is relatively resistant to in vivo degradation (e.g., via an exo- or endo-nuclease). Stabilization can be a function of length or secondary structure. Unmethylated CpG containing nucleic acid molecules that are tens to hundreds of kilobases long are relatively resistant to in vivo degradation. For shorter immunostimulatory nucleic acid molecules, secondary structure can stabilize and increase their effect. For example, if the 3′ end of a nucleic acid molecule has self-complementarity to an upstream region, so that it can fold back and form a sort of stem loop structure, then the nucleic acid molecule becomes stabilized and therefore exhibits more activity.

In certain embodiments, stabilized nucleic acid molecules of the instant invention have a modified backbone. It has been shown that modification of the oligonucleotide backbone provides enhanced activity of the CpG molecules of the invention when administered in vivo. CpG constructs, including at least two phosphorothioate linkages at the 5′ end of the oligodeoxyribonucleotide and multiple phosphorothioate linkages at the 3′ end, provided maximal activity and protected the oligodeoxyribonucleotide from degradation by intracellular exo- and endo-nucleases. Other modified oligodeoxyribonucleotides include phosphodiester modified oligodeoxyribonucleotide, combinations of phosphodiester, phosphorodithioate, and phosphorothioate oligodeoxyribonucleotide, methylphosphonate, methylphosphorothioate, phosphorodithioate, or methylphosphorothioate and combinations thereof. The phosphate backbone modification can occur at the 5′ end of the nucleic acid, for example at the first two nucleotides of the 5′ end of the nucleic acid. The phosphate backbone modification may occur at the 3′ end of the nucleic acid, for example at the last five nucleotides of the 3′ end of the nucleic acid. Nontraditional bases such as inosine and queosine, as well as acetyl-, thio- and similarly modified forms of adenine, cytidine, guanine, thymine, and uridine can also be included, which are not as easily recognized by endogenous endonucleases. Other stabilized nucleic acid molecules include: nonionic DNA analogs, such as alkyl- and aryl-phosphonates (in which the charged oxygen moiety is alkylated). Nucleic acid molecules that contain a diol, such as tetrahyleneglycol or hexaethyleneglycol, at either or both termini are also included.

DNA containing unmethylated CpG dinucleotide motifs in the context of certain flanking sequences has been found to be a potent stimulator of several types of immune cells in vitro. (Ballas, et al., J. Immunol. 157:1840 (1996); Cowdrey, et al., J. Immunol. 156:4570 (1996); Krieg, et al., Nature 374:546 (1995)) Depending on the flanking sequences, certain CpG motifs may be more immunostimulatory for B cell or T cell responses, and preferentially stimulate certain species. When a humoral response is desired, preferred immunostimulatory oligonucleotides comprising an unmethylated CpG motif will be those that preferentially stimulate a B cell response. When cell-mediated immunity is desired, preferred immunostimulatory oligonucleotides comprising at least one unmethylated CpG dinucleotide will be those that stimulate secretion of cytokines known to facilitate a CD8+ T cell response.

The immunostimulatory oligonucleotides of the invention may be chemically modified in a number of ways in order to stabilize the oligonucleotide against endogenous endonucleases. As used herein, these contain “synthetic phosphodiester backbones.” For example, the oligonucleotides may contain other than phosphodiester linkages in which the nucleotides at the 5′ end and/or 3′ end of the oligonucleotide have been replaced with any number of non-traditional bases or chemical groups, such as phosphorothioate-modified nucleotides. The immunostimulatory oligonucleotide comprising at least one unmethylated CpG dinucleotide may preferably be modified with at least one such phosphorothioate-modified nucleotide. Oligonucleotides with phosphorothioate-modified linkages may be prepared using methods well known in the field such as phosphoramidite (Agrawal, et al., Proc. Natl. Acad. Sci. 85:7079 (1988)) or H-phosphonate (Froehler, et al., Tetrahedron Lett. 27:5575 (1986)). Examples of other modifying chemical groups include alkylphosphonates, phosphorodithioates, alkylphosphorothioates, phosphoramidates, 2-O-methyls, carbamates, acetamidates, carboxymethyl esters, carbonates, and phosphate triesters. Oligonucleotides with these linkages can be prepared according to known methods (Goodchild, Chem. Rev. 90:543 (1990); Uhlmann, et al., Chem. Rev. 90:534 (1990); and Agrawal, et al., Trends Biotechnol. 10:152 (1992)). A “partially synthetic backbone” is a backbone where some of the oligonucleotides are modified, and a “completely synthetic backbone” is one where all of the oligonucleotides are modified. A “natural phosphodiester backbone” is one where the oligonucleotides have not been modified.

Other stabilized nucleic acid molecules include: nonionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated. Nucleic acid molecules which contain diol, such as tetraethyleneglycol or hexaethyleneglycol, at either or both termini have also been shown to be substantially resistant to nuclease degradation.

The term “vaccine composition” herein refers to a composition capable of producing an immune response. A vaccine composition, according to the invention, would produce immunity against disease in individuals.

Administration of the compositions of the present invention may be by parenteral, intravenous, intramuscular, subcutaneous, intranasal, oral, mucosal, intratumoral, or any other suitable means. In certain embodiments, the compositions are administered subcutaneously. The dosage administered may be dependent upon the age, weight, kind of concurrent treatment, if any, and nature of the antigen administered. The initial dose may be followed up with a booster dosage after a period of about four weeks to enhance the immunogenic response. Further booster dosages may also be administered. The composition may be given as a single injection of a mixed formulation of oligonucleotide and tumor lysate, or as separate injections given at the same region within a short period of time (e.g., 0-2 days). For example, the oligonucleotide(s) may be administered prior to the lysate. The composition may be administered multiple (e.g., 2, 3, 4 or 5) times at an interval of, e.g., about 1, 2, 3, 4, 5, 6 or 7, 14, or 21 days apart.

As used herein, the term “vector” refers to a nucleic acid molecule capable of transporting another nucleic acid to which it has been linked. Preferred vectors are those capable of autonomous replication and expression of nucleic acids to which they are linked (e.g. an episome). Vectors capable of directing the expression of genes to which they are operatively linked are referred to herein as “expression vectors.” In general, expression vectors of utility in recombinant DNA techniques are often in the form of “plasmids” which refer generally to circular double-stranded DNA loops which, in their vector form, are not bound to the chromosome. In the present specification, “plasmid” and “vector” are used interchangeably as the plasmid is the most commonly used form of vector. However, the invention is intended to include such other forms of expression vectors which serve equivalent functions and which become known in the art subsequently hereto.

A “subject” shall mean a human or vertebrate animal including a dog, cat, horse, cow, pig, sheep, goat, chicken, monkey, rat, and mouse. Nucleic acids containing an unmethylated CpG can be effective in any mammal, such as a human. Different nucleic acids containing an unmethylated CpG can cause optimal immune stimulation depending on the mammalian species. Thus an oligonucleotide causing optimal stimulation in humans may not cause optimal stimulation in a mouse. One of skill in the art can identify the optimal oligonucleotides useful for a particular mammalian species of interest.

The stimulation index of a particular immunostimulatory CpG ODN to effect an immune response can be tested in various immune cell assays. The stimulation index of the immune response can be assayed by measuring various immune parameters, e.g., measuring the antibody-forming capacity, number of lymphocyte subpopulations, mixed leukocyte response assay, lymphocyte proliferation assay. The stimulation of the immune response can also be measured in an assay to determine resistance to infection or tumor growth. Methods for measuring a stimulation index are well known to one of skill in the art For example, one assay is the incorporation of ³H uridine in a murine B cell culture, which has been contacted with a 20 pM of oligonucleotide for 20 h at 37° C. and has been pulsed with 1 pCi of ³H uridine; and harvested and counted 4 h later. The induction of secretion of a particular cytokine can also be used to assess the stimulation index. In one method, the stimulation index of the CpG ODN with regard to B-cell proliferation is at least about 5, at least about 10, at least about 15, or even at least about 20 (as described in detail in U.S. Pat. No. 6,239,116), while recognizing that there are differences in the stimulation index among individuals.

Immunostimulatory CpG nucleic acids should effect at least about 500 pg/ml of TNF-alpha, 15 pg/ml IFN-gamma, 70 pg/ml of GM-CSF 275 pg/ml of IL-6, 200 pg/ml IL-12, depending on the therapeutic indication. Other immunostimulatory CpG DNAs should effect at least about 10%, at least about 15%, or even at least about 20% YAC-1 cell specific lysis or at least about 30, at least about 35 or even at least about 40% 2C11 cell specific lysis. The CpG ODN of the invention stimulates cytokine production (e.g., IL-6, IL-12, IFN-gamma, TNF-alpha and GM-CSF) activate B cells and upregulate expression of MHC and B7 molecules.

The nucleic acid sequences of the invention useful for stimulating dendritic cells are those broadly described above. Exemplary sequences include sequences that comprise or consist of:

| | |
|---|---|
| 5′-TCCATGTCGCTCCTGATGCT-3′; | (SEQ ID NO:2) |
| 5′-TCCATGTCGTTCCTGATGCT-3′; | (SEQ ID NO:3) |
| 5′-TCGTCGTTTTGTCGTTTTGTCGTT-3′; | (SEQ ID NO:4) |
| 5′-TCGTCGTTGTCGTTGTCGTT-3′; | (SEQ ID NO:5) |
| 5′-TCGTCGTTGTCGTTTTGTCGTT-3′; | (SEQ ID NO:6) |
| 5′-GCGTGCGTTGTCGTTGTCGTT-3′; | (SEQ ID NO:7) |
| 5′-TGTCGTTTGTCGTTTGTCGTT-3′; | (SEQ ID NO:8) |
| 5′-TGTCGTTGTCGTTGTCGTT-3′; | (SEQ ID NO:9) |
| 5′-TCGTCGTCGTCGTT-3′; | (SEQ ID NO:10) |
| 5′-TCCTGTCGTTCCTTGTCGTT-3′; | (SEQ ID NO:11) |
| 5′-TCCTGTCGTTTTTTGTCGTT-3′; | (SEQ ID NO:12) |
| 5′-TCGTCGCTGTCTGCCCTTCTT-3′; | (SEQ ID NO:13) |
| 5′-TCGTCGCTGTTGTCGTTTCTT-3′; | (SEQ ID NO:14) |
| 5′-TCCATGACGTTCCTGACGTT-3′; | (SEQ ID NO:15) |
| 5′-GGGGGACGATCGTCGGGGGG-3′; | (SEQ ID NO:16) |
| 5′-TCGTCGTCGTTCGAACGACGTTGAT-3′; | (SEQ ID NO:17) |
| and | |
| 5′-TCGTCGTTTTGTCGTTTTGTCGTTGGGGG-3′. | (SEQ ID NO:18) |

In certain embodiments, CpG ODN can effect at least about 500 pg/ml of TNF-alpha, 15 pg/ml IFN-gamma, 70 pg/ml of GM-CSF 275 pg/ml of IL-6, 200 pg/ml IL-12, depending on the therapeutic indication. These cytokines can be measured by assays well known in the art. The ODNs listed above or other CpG ODN can effect at least about 10%, at least about 15%, or even at least about 20% YAC-1 cell specific lysis or at least about 30%, at least about 35%, or even at least about 40% 2C11 cell specific lysis, in assays well known in the art.

The term “polynucleotide” or “nucleic acid sequence” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA that is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. The term includes single and double stranded forms of DNA.

Methods for Making Tumor Lysates

Tumor lysates are made by extracting a sample of the tumor to be treated from the subject. The tumor cells are then lysed. Methods of making effective tumor lysates include, but are not limited to, freeze thaw method, sonication, microwave, boiling, high heat, detergent or chemical-based cell lysis, electric or current-based lysis, and other physical methods, such as extreme force.

In certain embodiments, such as when a glioma is to be treated, EGF receptor VIII variant and IL-13 receptor alpha-2, which are glioma specific receptors (or expression vectors encoding these proteins), may be added to the tumor lysate.

Tumor Lysis Agents

Tumor lysis agents include those agents that are known to lyse tumor cells in vivo. Examples include, but are not limited to chemotherapeutic agents or biological toxins. Tumor lysis agents include but are not limited to temozolomide (Temodar®), Temodar, Carboplatin, Doxyrubicin, or a replication competent CMV virus. In certain embodiments, the tumor lysis agent is diphtheria toxin.

Methods for Making Immunostimulatory Nucleic Acids

For use in the instant invention, nucleic acids can be synthesized de novo using any of a number of procedures well known in the art. For example, the B-cyanoethyl phosphoramidite method (S. L. Beaucage and M. H. Caruthers, 1981, Tet. Let. 22:1859); nucleoside H-phosphonate method (Garegg, et al., 1986, Tet. Let. 27:4051-4051; Froehler, et al., 1986, Nucl. Acid. Res. 14:5399-5407; Garegg, et al., 1986, Tet. Let. 27:4055-4058, Gaffney, et al., 1988), Tet. Let. 29:2619-2622. These chemistries can be performed by a variety of automated oligonucleotide synthesizers available in the market. Alternatively, oligonucleotides can be prepared from existing nucleic acid sequences (e.g., genomic or cDNA) using known techniques, such as those employing restriction enzymes, exonucleases or endonucleases.

For use in vivo, nucleic acids are preferably relatively resistant to degradation (e.g., via endo- and exo-nucleases). Secondary structures, such as stem loops, can stabilize nucleic acids against degradation. Alternatively, nucleic acid stabilization can be accomplished via phosphate backbone modifications. A stabilized nucleic acid can be accomplished via phosphate backbone modifications. A stabilized nucleic acid has at least a partial phosphorothioate modified backbone. Phosphorothioates may be synthesized using automated techniques employing either phosphoramidate or H-phosphonate chemistries. Aryl- and alkyl-phosphonates can be made for example as described in U.S. Pat. No. 4,469,863; and alkylphosphotriesters (in which the charged oxygen moiety is alkylated as described in U.S. Pat. No. 5,023,243 and European Patent No. 092,574) can be prepared by automated solid phase synthesis using commercially available reagents. Methods for making other DNA backbone modifications and substitutions have been described (Uhlmann, E. and Peyman, A., 1990, Chem. Rev. 90:544; Goodchild, J., 1990, Bioconjugate Chem. 1:165). 2′-O-methyl nucleic acids with CpG motifs also cause immune activation, as do ethoxy-modified CpG nucleic acids. In fact, no backbone modifications have been found that completely abolish the CpG effect, although it is greatly reduced by replacing the C with a 5-methyl C.

Therapeutic Uses of Immunostimulatory Nucleic Acid Molecules

The tumors or cancers to be treated and/or used to generate a tumor lysate may be a solid tumors or hematological cancers. The tumor to be treated using the method of the present invention may be a solid tumor and may be cancerous. In particular, the solid tumor may be a lung tumor, a melanoma, a mesothelioma, a mediastinum tumor, esophagal tumor, stomach tumor, pancreal tumor, renal tumor, liver tumor, hepatobiliary system tumor, small intestine tumor, colon tumor, rectum tumor, anal tumor, kidney tumor, ureter tumor, bladder tumor, prostate tumor, urethral tumor, testicular tumor, gynecological organ tumor, ovarian tumor, breast tumor, endocrine system tumor, or central nervous system (e.g., brain) tumor. The cancers to be treated may be a hematological cancer, such as a lymphoma, leukemia, pancreatic cancer, or macroglobulinema.

In one embodiment, the invention provides a method for stimulating an immune response in a subject by administering a therapeutically effective amount of a nucleic acid sequence containing at least one unmethylated CpG dinucleotide mixed with a tumor cell lysate. This invention provides administering to a subject having or at risk of having a tumor, a therapeutically effective dose of a pharmaceutical composition containing the compounds of the present invention and a pharmaceutically acceptable carrier. “Administering” the pharmaceutical composition of the present invention may be accomplished by any means known to the skilled artisan.

Immunostimulatory oligonucleotides and unmethylated CpG containing vaccines, which directly activate lymphocytes and co-stimulate an antigen-specific response, are fundamentally different from conventional adjuvants (e.g., aluminum precipitates), which are inert when injected alone and are thought to work through absorbing the antigen and thereby presenting it more effectively to immune cells. Further, conventional adjuvants only work for certain antigens, only induce an antibody (humoral) immune response (T_{H}2), and are very poor at inducing cellular immune responses (T_{H}1).

An immunostimulatory oligonucleotide can be administered prior to, along with or after administration of a chemotherapy or immunotherapy to increase the responsiveness of the malignant cells to subsequent chemotherapy or immunotherapy or to speed the recovery of the bone marrow through induction of restorative cytokines such as GM-CSF. CpG nucleic acids also increase natural killer cell lytic activity and antibody dependent cellular cytotoxicity (ADCC). Induction of NK activity and ADCC may likewise be beneficial in cancer immunotherapy, alone or in conjunction with other treatments.

For use in therapy, an effective amount of an appropriate immunostimulatory nucleic acid molecule formulated as a delivery complex along with a tumor cell lysate can be administered to a subject by any mode allowing the oligonucleotide to be taken up by the appropriate target cells (e.g., dendritic cells). Routes of administration include oral and transdermal (e.g., via a patch). Examples of other routes of administration include injection (subcutaneous, intravenous, parenteral, intraperitoneal, intrathecal, etc.). The injection can be in a bolus or a continuous infusion.

A nucleic acid delivery complex can be administered in conjunction with a pharmaceutically acceptable carrier. As used herein, the phrase “pharmaceutically acceptable carrier” is intended to include substances that can be co-administered with a nucleic acid or a nucleic acid delivery complex and allows the nucleic acid to perform its indicated function. Examples of such carriers include solutions, solvents, dispersion media, delay agents, emulsions and the like. The use of such media for pharmaceutically active substances are well known in the art. Any other conventional carrier suitable for use with the nucleic acids falls within the scope of the instant invention.

The term “effective amount” of a nucleic acid molecule refers to the amount necessary or sufficient to realize a desired biologic effect. For example, an effective amount of a nucleic acid containing at least one unmethylated CpG for inducing an immune reaction could be that amount necessary to eliminate a tumor or cancer. The effective amount for any particular application can vary depending on such factors as the disease or condition being treated, the particular nucleic acid being administered (e.g., the number of unmethylated CpG motifs or their location in the nucleic acid), the size of the subject, or the severity of the condition. One of ordinary skill in the art can empirically determine the effective amount of a particular oligonucleotide without necessitating undue experimentation.

The compositions of the invention, including isolated CpG nucleic acid molecules, tumor lysates, and mixtures thereof are administered in pharmaceutically acceptable compositions. The compositions may be administered by bolus injection, continuous infusion, sustained release from implants, aerosol, or any other suitable technique known in the art.

The pharmaceutical compositions according to the invention are in general administered topically, intravenously, orally, parenterally or as implants, and even rectal use is possible in principle. Suitable solid or liquid pharmaceutical preparation forms are, for example, granules, powders, tablets, coated tablets, (micro) capsules, suppositories, syrups, emulsions, suspensions, creams, aerosols, drops or injectable solution in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249: 1527-1533, 1990, which is incorporated herein by reference.

The pharmaceutical compositions may be prepared and administered in dose units.

Solid dose units are tablets, capsules and suppositories. For treatment of a patient, depending on activity of the compound, manner of administration, nature and severity of the disorder, age and body weight of the patient, different doses are necessary. Under certain circumstances, however, higher or lower doses may be appropriate. The administration of the dose can be carried out both by single administration in the form of an individual dose unit or else several smaller dose units and also by multiple administrations of subdivided doses at specific intervals.

The pharmaceutical compositions according to the invention may be administered locally or systemically. By “therapeutically effective dose” is meant the quantity of a compound according to the invention necessary to prevent, to cure or at least partially arrest the symptoms and complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Reminpton's Pharmaceutical Sciences, 17th ed., Mack Publishing Co., Easton, Pa., 1990, each of which is herein incorporated by reference.

Adjuvants

An oligonucleotide containing at least one unmethylated CpG can be used alone to activate the immune response or can be administered in combination with an adjuvant. An “adjuvant” is any molecule or compound that nonspecifically stimulate the humoral and/or cellular immune response. They are considered to be nonspecific because they only produce an immune response in the presence of an antigen. Adjuvants allow much smaller doses of antigen to be used and are essential to inducing a strong antibody response to soluble antigens. For example, when the oligonucleotide containing at least one unmethylated CpG is administered in conjunction with another adjuvant, the oligonucleotide can be administered before, after, and/or simultaneously with the other adjuvant. The oligonucleotide containing at least one unmethylated CpG can have an additional efficacy in addition to its ability to activate the immune response.

Stimulation of Cytokines

The invention further provides a method of modulating the level of a cytokine. The term “modulate” envisions the suppression of expression of a particular cytokine when it is overexpressed, or augmentation of the expression of a particular cytokine when it is underexpressed. Modulation of a particular cytokine can occur locally or systemically.

It is believed that the CpG oligonucleotides do not directly activate purified NK cells, but rather render them competent to respond to IL-12 with a marked increase in their IFN-y production. By inducing IL-12 production and the subsequent increased IFN-y secretion by NK cells, the immunostimulatory nucleic acids also promote a T_{H}1 type immune response. No direct activation of proliferation or cytokine secretion by highly purified T cells has been found. Cytokine profiles determine T cell regulatory and effector functions in immune responses.

Cytokines also play a role in directing the T cell response. Helper (CD4+) T cells orchestrate the immune response of mammals through production of soluble factors that act on other immune system cells, including B and other T cells. Most mature CD4+ T helper cells express one of two cytokine profiles: T_{H}1 or T_{H}2. T_{H}1 cells secrete IL-2, IL-3, IFN-gamma, TNF-P, GM-CSF and high levels of TNF-alpha. T_{H}2 cells express IL-3, IL-4, IL-5, IL-6, IL-9, IL-10, IL-13, GM-CSF and low levels of TNF-alpha. The T_{H}1 subset promotes delayed-type hypersensitivity, cell-mediated immunity, and immunoglobulin class switching to IgG2a. The T_{H}2 subset induces humoral immunity by activating B cells, promoting antibody production, and inducing class switching to IgG, and IgE.

Several factors have been shown to influence commitment to T_{H}1 or T_{H}2 profiles. The best characterized regulators are cytokines. IL-12 and IFN-gamma are positive T_{H}1 and negative T_{H}2 regulators. IL-12 promotes IFN-gamma production, and IFN-gamma provides positive feedback for IL-12. IL-4 and IL-10 appear to be required for the establishment of the T_{H}2 cytokine profile and to down-regulate T_{H}1 cytokine production. The effects of IL-4 are in some cases dominant over those of IL-12. IL-13 was shown to inhibit expression of inflammatory cytokines, including IL-12 and TNF-a by LPS-induced monocytes, in a way similar to IL-4. The IL-12 p40 homodimer binds to the IL-12 receptor and antagonizes IL-12 biological activity; thus it blocks the pro-T_{H}1 effects of IL-12.

Expression Vectors

The term “polynucleotide” or “nucleic acid sequence” refers to a polymeric form of nucleotides at least 10 bases in length. By “isolated polynucleotide” is meant a polynucleotide that is not immediately contiguous with both of the coding sequences with which it is immediately contiguous (one on the 5′ end and one on the 3′ end) in the naturally occurring genome of the organism from which it is derived. The term therefore includes, for example, a recombinant DNA that is incorporated into a vector; into an autonomously replicating plasmid or virus; or into the genomic DNA of a prokaryote or eukaryote, or which exists as a separate molecule (e.g., a cDNA) independent of other sequences. The nucleotides of the invention can be ribonucleotides, deoxyribonucleotides, or modified forms of either nucleotide. The term includes single and double stranded forms of DNA.

In the present invention, the polynucleotide sequences encoding interferon-gamma (INF-gamma) may be inserted into an expression vector. The term “expression vector” refers to a plasmid, virus or other vehicle known in the art that has been manipulated by insertion or incorporation of the genetic sequences encoding the antigenic polypeptide.

Polynucleotide sequences that encode the INF-gamma can be operatively linked to expression control sequences. “Operatively linked” refers to a juxtaposition wherein the components so described are in a relationship permitting them to function in their intended manner. An expression control sequence operatively linked to a coding sequence is ligated such that expression of the coding sequence is achieved under conditions compatible with the expression control sequences. As used herein, the term “expression control sequences” refers to nucleic acid sequences that regulate the expression of a nucleic acid sequence to which it is operatively linked. Expression control sequences are operatively linked to a nucleic acid sequence when the expression control sequences control and regulate the transcription and, as appropriate, translation of the nucleic acid sequence. Thus expression control sequences can include appropriate promoters, enhancers, transcription terminators, as start codon (i.e., ATG) in front of a protein-encoding gene, splicing signal for introns, maintenance of the correct reading frame of that gene to permit proper translation of mRNA, and stop codons. The term “control sequences” is intended to included, at a minimum, components whose presence can influence expression, and can also include additional components whose presence is advantageous, for example, leader sequences and fusion partner sequences.

Expression control sequences can include a promoter. By “promoter” is meant minimal sequence sufficient to direct transcription. Also included in the invention are those promoter elements that are sufficient to render promoter-dependent gene expression controllable for cell-type specific, tissue-specific, or inducible by external signals or agents; such elements may be located in the 5′ or 3′ regions of the gene. Both constitutive and inducible promoters are included in the invention.

Promoters derived from the genome of mammalian cells (e.g., metallothionein promoter) or from mammalian viruses (e.g., the retrovirus long terminal repeat; the adenovirus late promoter; the vaccinia virus 7.5K promoter), cytomegalovirus (CMV), or hepatitis B virus (HBV) may be used. Promoters produced by recombinant DNA or synthetic techniques may also be used to provide for transcription of the nucleic acid sequences of the invention.

Methods for Introducing Genetic Material into Cells

The exogenous genetic material (e.g., an expression vector encoding INF-gamma) is introduced into the cell ex vivo or in vivo by genetic transfer methods, such as transfection or transduction, to provide a genetically modified cell. Various expression vectors (i.e., vehicles for facilitating delivery of exogenous genetic material into a target cell) are known to one of ordinary skill in the art.

As used herein, “transfection of cells” refers to the acquisition by a cell of new genetic material by incorporation of added DNA. Thus, transfection refers to the insertion of nucleic acid into a cell using physical or chemical methods. Several transfection techniques are known to those of ordinary skill in the art including: calcium phosphate DNA co-precipitation (Methods in Molecular Biology, Vol. 7, Gene Transfer and Expression Protocols, Ed. E. J. Murray, Humana Press (1991)); DEAE-dextran (supra); electroporation (supra); cationic liposome-mediated transfection (supra); and tungsten particle-faciliated microparticle bombardment (Johnston, S. A., Nature 346:776-777 (1990)). Strontium phosphate DNA co-precipitation (Brash D. E. et al. Molec. Cell. Biol. 7:2031-2034 (1987) is another possible transfection method.

In contrast, “transduction of cells” refers to the process of transferring nucleic acid into a cell using a DNA or RNA virus. A RNA virus (i.e., a retrovirus) for transferring a nucleic acid into a cell is referred to herein as a transducing chimeric retrovirus. Exogenous genetic material contained within the retrovirus is incorporated into the genome of the transduced cell. A cell that has been transduced with a chimeric DNA virus (e.g., an adenovirus carrying a cDNA encoding a therapeutic agent), will not have the exogenous genetic material incorporated into its genome but will be capable of expressing the exogenous genetic material that is retained extrachromosomally within the cell.

Typically, the exogenous genetic material includes the heterologous gene (usually in the form of a cDNA comprising the exons coding for the therapeutic protein) together with a promoter to control transcription of the new gene. The promoter characteristically has a specific nucleotide sequence necessary to initiate transcription. Optionally, the exogenous genetic material further includes additional sequences (i.e., enhancers) required to obtain the desired gene transcription activity. For the purpose of this discussion an “enhancer” is simply any non-translated DNA sequence which works contiguous with the coding sequence (in cis) to change the basal transcription level dictated by the promoter. The exogenous genetic material may introduced into the cell genome immediately downstream from the promoter so that the promoter and coding sequence are operatively linked so as to permit transcription of the coding sequence. A retroviral expression vector may include an exogenous promoter element to control transcription of the inserted exogenous gene. Such exogenous promoters include both constitutive and inducible promoters.

Naturally-occurring constitutive promoters control the expression of essential cell functions. As a result, a gene under the control of a constitutive promoter is expressed under all conditions of cell growth. Exemplary constitutive promoters include the promoters for the following genes which encode certain constitutive or “housekeeping” functions: hypoxanthine phosphoribosyl transferase (HPRT), dihydrofolate reductase (DHFR) (Scharfmann et al., Proc. Natl. Acad. Sci. USA 88: 4626-4630 (1991)), adenosine deaminase, phosphoglycerol kinase (PGK), pyruvate kinase, phosphoglycerol mutase, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

Genes that are under the control of inducible promoters are expressed only or to a greater degree, in the presence of an inducing agent (e.g., transcription under control of the metallothionein promoter is greatly increased in presence of certain metal ions). Inducible promoters include responsive elements (REs) which stimulate transcription when their inducing factors are bound. For example, there are REs for serum factors, steroid hormones, retinoic acid and cyclic AMP. Promoters containing a particular RE can be chosen in order to obtain an inducible response and in some cases, the RE itself may be attached to a different promoter, thereby conferring inducibility to the recombinant gene. Thus, by selecting the appropriate promoter (constitutive versus inducible; strong versus weak), it is possible to control both the existence and level of expression of a therapeutic agent in the genetically modified cell. If the gene encoding the therapeutic agent is under the control of an inducible promoter, delivery of the therapeutic agent in situ is triggered by exposing the genetically modified cell in situ to conditions for permitting transcription of the therapeutic agent, e.g., by intraperitoneal injection of specific inducers of the inducible promoters which control transcription of the agent. For example, in situ expression by genetically modified cells of a therapeutic agent encoded by a gene under the control of the metallothionein promoter is enhanced by contacting the genetically modified cells with a solution containing the appropriate (i.e., inducing) metal ions in situ.

Accordingly, the amount of therapeutic agent that is delivered in situ is regulated by controlling such factors as: (1) the nature of the promoter used to direct transcription of the inserted gene, (i.e., whether the promoter is constitutive or inducible, strong or weak); (2) the number of copies of the exogenous gene that are inserted into the cell; (3) the number of transduced/transfected cells that are administered (e.g., implanted) to the patient; (4) the size of the implant (e.g., graft or encapsulated expression system); (5) the number of implants; (6) the length of time the transduced/transfected cells or implants are left in place; and (7) the production rate of the therapeutic agent by the genetically modified cell. Selection and optimization of these factors for delivery of a therapeutically effective dose of a particular therapeutic agent is deemed to be within the scope of one of ordinary skill in the art without undue experimentation, taking into account the above-disclosed factors and the clinical profile of the patient.

In addition to at least one promoter and at least one heterologous nucleic acid encoding the therapeutic agent, the expression vector may include a selection gene, for example, a neomycin resistance gene, for facilitating selection of cells that have been transfected or transduced with the expression vector. Alternatively, the cells are transfected with two or more expression vectors, at least one vector containing the gene(s) encoding the therapeutic agent(s), the other vector containing a selection gene. The selection of a suitable promoter, enhancer, selection gene and/or signal sequence is deemed to be within the scope of one of ordinary skill in the art without undue experimentation.

The therapeutic agent can be targeted for delivery to an extracellular, intracellular or membrane location. If it is desirable for the gene product to be secreted from the cells, the expression vector is designed to include an appropriate secretion “signal” sequence for secreting the therapeutic gene product from the cell to the extracellular milieu. If it is desirable for the gene product to be retained within the cell, this secretion signal sequence is omitted. In a similar manner, the expression vector can be constructed to include “retention” signal sequences for anchoring the therapeutic agent within the cell plasma membrane. For example, all membrane proteins have hydrophobic transmembrane regions, which stop translocation of the protein in the membrane and do not allow the protein to be secreted. The construction of an expression vector including signal sequences for targeting a gene product to a particular location is deemed to be within the scope of one of ordinary skill in the art without the need for undue experimentation.

The selection and optimization of a particular expression vector for expressing a specific gene product in an isolated cell is accomplished by obtaining the gene, potentially with one or more appropriate control regions (e.g., promoter, insertion sequence); preparing a vector construct comprising the vector into which is inserted the gene; transfecting or transducing cultured cells in vitro with the vector construct; and determining whether the gene product is present in the cultured cells.

In one embodiment, vectors for cell gene therapy are viruses, such as replication-deficient viruses (described in detail below). Exemplary viral vectors are derived from: Harvey Sarcoma virus; ROUS Sarcoma virus, (MPSV); Moloney murine leukemia virus and DNA viruses (e.g., adenovirus) (Ternin, H., “Retrovirus vectors for gene transfer”, in Gene Transfer, Kucherlapati R, Ed., pp 149-187, Plenum, (1986)).

Replication-deficient retroviruses, including the recombinant lentivirus vectors, are neither capable of directing synthesis of virion proteins or making infectious particles. Accordingly, these genetically altered retroviral expression vectors have general utility for high-efficiency transduction of genes in cultured cells, and specific utility for use in the method of the present invention. The lentiviruses, with their ability to transduce nondividing cells, have general utility for transduction of hepatocytes, cells in cerebrum, cerebellum and spinal cord, and also muscle and other slowly or non-dividing cells. Such retroviruses further have utility for the efficient transduction of genes into cells in vivo. Retroviruses have been used extensively for transferring genetic material into cells. Standard protocols for producing replication-deficient retroviruses (including the steps of incorporation of exogenous genetic material into a plasmid, transfection of a packaging cell line with plasmid, production of recombinant retroviruses by the packaging cell line, collection of viral particles from tissue culture media, and infection of the target cells with the viral particles) are provided in Kriegler, M. Gene Transfer and Expression, A Laboratory Manual, W. H. Freeman Co, New York, (1990) and Murray, E. J., ed. Methods in Molecular Biology., Vol. 7, Humana Press Inc., Clifton, N.J., (1991).

The major advantage of using retroviruses, including lentiviruses, for gene therapy is that the viruses insert the gene encoding the therapeutic agent into the host cell genome, thereby permitting the exogenous genetic material to be passed on to the progeny of the cell when it divides. In addition, gene promoter sequences in the LTR region have been reported to enhance expression of an inserted coding sequence in a variety of cell types (see e.g., Hilberg et al., Proc. Natl. Acad. Sci. USA 84:5232-5236 (1987); Holland et al., Proc. Natl. Acad. Sci. USA 84:8662-8666 (1987); Valerio et al., Gene 84:419-427 (1989). The major disadvantages of using a retrovirus expression vector are (1) insertional mutagenesis, i.e., the insertion of the therapeutic gene into an undesirable position in the target cell genome which, for example, leads to unregulated cell growth and (2) the need for target cell proliferation in order for the therapeutic gene carried by the vector to be integrated into the target genome (Miller, D. G., et al., Mol. Cell. Biol. 10:4239-4242 (1990)). While proliferation of the target cell is readily achieved in vitro, proliferation of many potential target cells in vivo is very low.

Yet another viral candidate useful as an expression vector for transformation of cells is the adenovirus, a double-stranded DNA virus. The adenovirus is frequently responsible for respiratory tract infections in humans and thus appears to have avidity for the epithelium of the respiratory tract (Straus, S., The Adenovirus, H. S. Ginsberg, Editor, Plenum Press, New York, P. 451-496 (1984)). Moreover, the adenovirus is infective in a wide range of cell types, including, for example, muscle and endothelial cells (Larrick, J. W. and Burck, K. L., Gene Therapy. Application of Molecular Biology, Elsevier Science Publishing Co., Inc., New York, p. 71-104 (1991)). The adenovirus also has been used as an expression vector in muscle cells in vivo (Quantin, B., et al., Proc. Natl. Acad. Sci. USA 89:2581-2584 (1992)).

Like the retrovirus, the adenovirus genome is adaptable for use as an expression vector for gene therapy, i.e., by removing the genetic information that controls production of the virus itself (Rosenfeld, M. A., et al., Science 252:431434 (1991)). Because the adenovirus functions in an extrachromosomal fashion, the recombinant adenovirus does not have the theoretical problem of insertional mutagenesis.

Finally, a third virus family adaptable for an expression vector for gene therapy are the recombinant adeno-associated viruses, specifically those based on AAV2, AAV4 and AAV5 (Davidson et al, PNAS 97:3428-3432 (2000)).

Thus, as will be apparent to one of ordinary skill in the art, a variety of suitable viral expression vectors are available for transferring exogenous genetic material into cells. The selection of an appropriate expression vector to express a therapeutic agent for a particular condition amenable to gene replacement therapy and the optimization of the conditions for insertion of the selected expression vector into the cell, are within the scope of one of ordinary skill in the art without the need for undue experimentation.

In an alternative embodiment, the expression vector is in the form of a plasmid (such as the Sleeping Beauty plasmid), which is transferred into the target cells by one of a variety of methods: physical (e.g., microinjection (Capecchi, M. R., Cell 22:479-488 (1980)), electroporation (Andreason, G. L. and Evans, G. A. Biotechniques 6:650-660 (1988), scrape loading, microparticle bombardment (Johnston, S. A., Nature 346:776-777 (1990)) or by cellular uptake as a chemical complex (e.g., calcium or strontium co-precipitation, complexation with lipid, complexation with ligand) (Methods in Molecular Biology, Vol. 7, Gene Transfer and Expression Protocols, Ed. E. J. Murray, Humana Press (1991)). Several commercial products are available for cationic liposome complexation including Lipofectin™ (Gibco-BRL, Gaithersburg, Md.) (Felgner, P. L., et al., Proc. Natl. Acad. Sci. 84:7413-7417 (1987)) and Transfectam™ (Promega, Madison, Wis.) (Behr, J. P., et al., Proc. Natl. Acad. Sci. USA 86:6982-6986 (1989); Loeffler, J. P., et al., J. Neurochem. 54:1812-1815 (1990)). However, the efficiency of transfection by these methods is highly dependent on the nature of the target cell and accordingly, the conditions for optimal transfection of nucleic acids into cells using the above-mentioned procedures must be optimized. Such optimization is within the scope of one of ordinary skill in the art without the need for undue experimentation. One protocol for using nonviral vectors for cancer gene therapy is provided in Ohlfest et al., Current Gene Therapy, 2005, 5:629-641, which is incorporated by reference in its entirety herein.

The instant invention also provides various methods for making and using the above-described genetically-modified cells. In particular, the invention provides a method for genetically modifying cell(s) of a mammalian recipient ex vivo and administering the genetically modified cells to the mammalian recipient. In one embodiment for ex vivo gene therapy, the cells are autologous cells, i.e., cells isolated from the mammalian recipient. As used herein, the term “isolated” means a cell or a plurality of cells that have been removed from their naturally-occurring in vivo location. Methods for removing cells from a patient, as well as methods for maintaining the isolated cells in culture are known to those of ordinary skill in the art.

The instant invention also provides methods for genetically modifying cells of a mammalian recipient in vivo. According to one embodiment, the method comprises introducing an expression vector for expressing a heterologous gene product into cells of the mammalian recipient in situ by, for example, injecting the vector into the recipient.

In one embodiment, the preparation of genetically modified cells contains an amount of cells sufficient to deliver a therapeutically effective dose of the therapeutic agent to the recipient in situ. The determination of a therapeutically effective dose of a specific therapeutic agent for a known condition is within the scope of one of ordinary skill in the art without the need for undue experimentation. Thus, in determining the effective dose, one of ordinary skill would consider the condition of the patient, the severity of the condition, as well as the results of clinical studies of the specific therapeutic agent being administered.

If the genetically modified cells are not already present in a pharmaceutically acceptable carrier they are placed in such a carrier prior to administration to the recipient. Such pharmaceutically acceptable carriers include, for example, isotonic saline and other buffers as appropriate to the patient and therapy.

The following examples are intended to illustrate but not to limit the invention in any manner, shape, or form, either explicitly or implicitly. While they are typical of those that might be used, other procedures, methodologies, or techniques known to those skilled in the art may alternatively be used.

### Example 1

### Bioluminescent Tumor Imaging

Bioluminescent tumor imaging has become a powerful tool to measure tumor location, size, and viability in vivo. Tumor cell lines that are genetically engineered to express luciferase are injected into rodents to form tumors; the amount of light emitted from the tumor cells is directly proportional to the amount of viable tumor cells. Bioluminescent tumor imaging has been used to assess the growth of intracranially implanted glioma cells in mice. A strong linear correlation exists between tumor size and emitted photons (R²=0.99). Luciferase-stable U87 glioma cells were used previously to visualize tumor regression in response to anti-angiogenic gene therapy. Thus, U87-Luc cells are suitable to quantify intracranial tumor burden in nude mice by measuring photons; tumor regression is determined in response to gene therapy in vivo and in real-time.

### Example 2

### INF-Gamma is an Effective Gene Therapy for Cancer

Mice bearing intracranial GL261-Luc tumors were treated with gene transfer of INF-γ with and without SB-encoding DNA, and observed an SB-dependent increase in animal survival. This result is consistent to data obtained in the nude mouse U87-Luc glioma model that showed SB-encoding DNA was required for long-term survival.

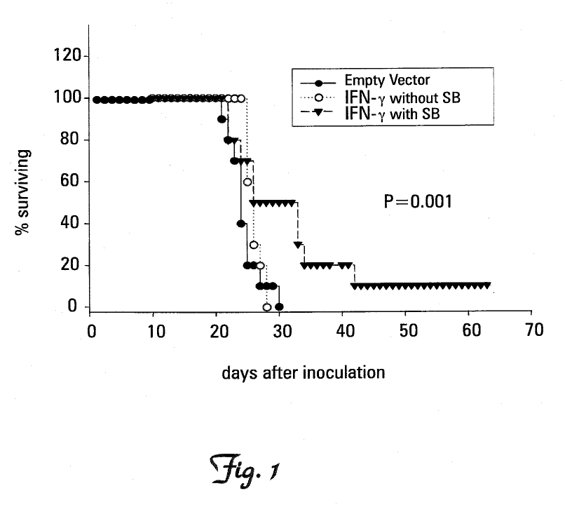

INF-gamma is an effective therapy for brain tumors. (FIG. 1) Human brain tumor stem cells are invisible to the immune system by downregulation of MHCI and activating ligands for natural killer cells, but incubation of these cancer stem cells in INF-gamma restores their immunogenecity. Mice bearing large intracranial brain tumors are effectively treated by intratumoral infusions of Sleeping Beauty-based plasmid DNA vectors encoding INF-gamma. INF-gamma gene therapy could be used to treat a variety of cancers.

INF-γ gene transfer increased survival with SB. C57BL/6 mice bearing i.c. GL261 gliomas were treated by intratumoral injection of 2.5 μg of DNA/PEI complexes containing an INF-γ transposon with or without SB-encoding DNA or saline (control; n=10/group). Only mice treated with INF-γ and SB exhibited a significant increase in survival by log rank (Mantel Cox) statistical analysis (p=0.001). Between 10-70% of the mice are completely cured. The mice that are not cured live significantly longer than non-treated controls.

As described below, when CpG+tumor lysate treatment was combined with intratumoral gene therapy (i.e., a vector encoding interferon-gamma), a 70% cure rate was achieved. The lysate+CpG treatment alone yields at least a 10-20% cure rate.

Methods

Tumor Inoculation/Stereotactic Surgery Protocol: Adult C57BL/6 mice were given intracranial gliomas by stereotactic injection of 10,000 GL261-Luc cells in 1 μl into the right striatum (0.5 AP, 1.8 ML, 3.0 DV mm from bregma) to establish a luciferase-stable glioma. For all stereotactic surgery, mice were anesthetized by i.p. injection with ketamine and secured into a stereotactic frame. Hair was shaved from the scalp and the skin was prepared for aseptic surgery with propidium iodine. A small incision was made with scalpel, followed by a small bur hole with a mircodrill in the skull at the appropriate coordinates. After injection of cells (or DNA) the bur hole was filled with bone wax, the incision sutured, and the mice were monitored until they regain consciousness.

Gene Therapy Protocol: Three days after inoculation, treatment of gliomas by gene therapy began. Transposon-plasmid DNA (1.65 μg/vector administered) complexed in PEI was delivered directly into the identical coordinates where the tumor was implanted in a 5-μl volume over twenty minutes (i.e., CED-mediated intratumoral delivery). The final volume of vector administered was always 5 μl, regardless of the number of genes.

Three control groups were included to control for vector (empty vector) or no DNA (saline), or episomal DNA (no SB-transposase; conducted last using most efficacious combinations of genes delivered with SB). Ten total mice in each group were treated with these vectors; but in order to ensure feasibility, five mice/group were treated and the experiment was repeated to get a final sample size of 10 mice/group. All mice were weighed two times per week. Tumor growth and/or regression is measured one time every week by luciferase in vivo imaging. All mice were monitored every day for signs of neurological abnormalities or morbidity (hunched posture, tremors, inactivity, etc). Any mouse that became moribund was humanely euthanized and a full necropsy was performed and the brains processed for histological analysis.

Data Analysis

Animal survival is the definitive measure of efficacy; gene therapy combinations that cause the greatest extension of survival time compared to empty vector and saline-treated controls is considered effective. The three control groups (saline, empty vector, and No-SB/short-term expression) die from tumor burden first. Significant extension in survival was considered as p≦0.05 by long-rank (Mantel-Cox) statistical analysis. Significant differences between the efficacy of different gene combination treatments was determined identically to survival (p≦0.05 between two groups). Reduction in tumor growth rate measured by luciferase imaging in vivo and overall health determined by body weight allows us to determine the time dynamics of tumor growth and assess health improvements in response to gene therapy.

### Example 3

### Tumor Lysate and CpG ODNs as a Therapy for Cancer

Due to the short-comings of the current procedures for treating cancer, a dramatically improved process has been developed. Tumor cells were colleted and lysed to make a “tumor lysate.” The tumor lysate contains tumor-specific antigens that the immune system can recognize. In order activate the immune system to expand killer T cells that will track down cells expressing these antigens (e.g., tumor cells growing in patient), CpG ODNs were mixed with the tumor lysate.

The tumor lysate/CpG mixture was prepared as follows: 2×10⁶ glioma cells (GL261-Luc cells) were resuspended in 50 μl saline and subjected to four series of freeze thaws by placing the cells in a ⅕ mL tube and freezing at −80° C., then thawing at 37° C. in a water bath. After freeze thawing, 100 μg of CpG ODN 2006 was pipetted into the tumor lysate.

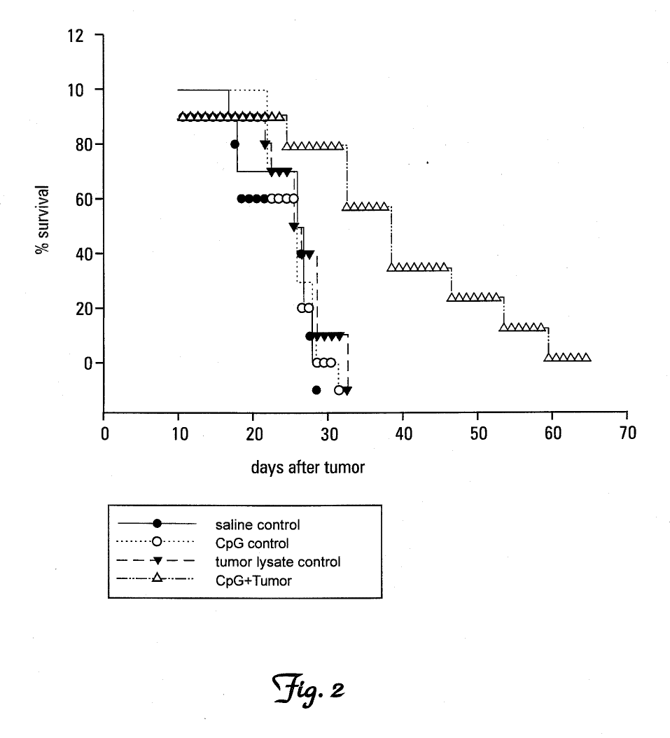

This mixture was then immediately injected subcutaneously in mice bearing established intracranial brain tumors (glioblastoma). This injection was given three times. The results showed that this lysate+CpG treatment yielded a 10-20% cure rate. Mice that were not cured lived significantly longer than the non-treated controls. (FIG. 2)

### Example 4

### INF-Gamma in Combination with Tumor Lysate and CpG as a Therapy for Cancer

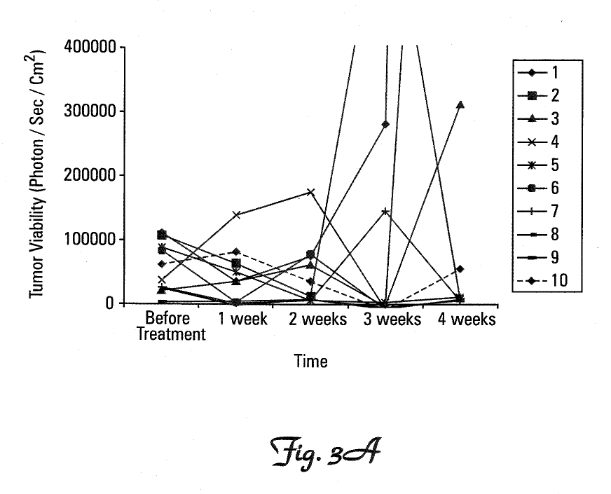

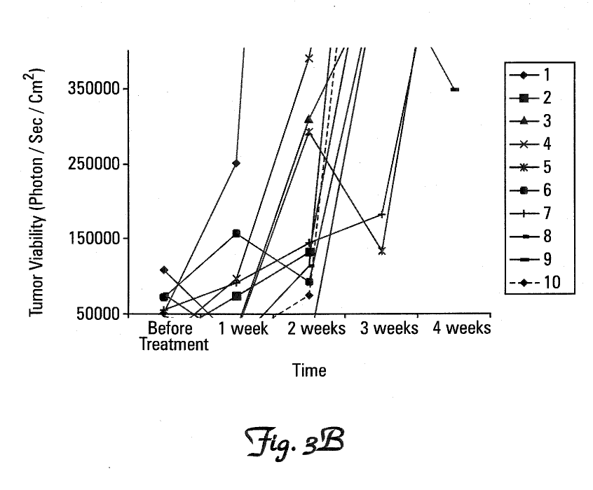

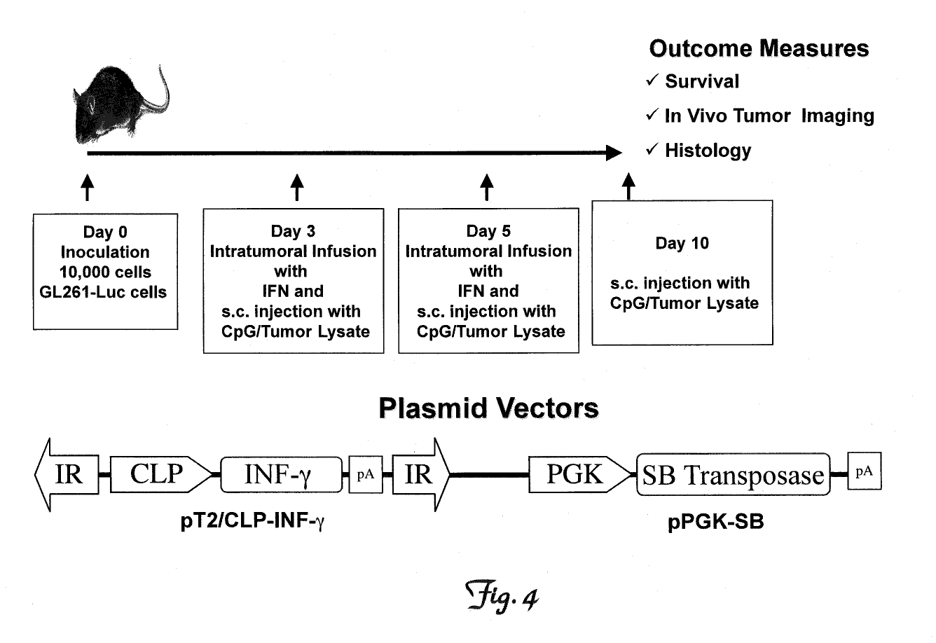

When the tumor lysate/CpG mixture procedure described above was combined with intratumoral gene therapy with a vector encoding interferon-gamma, an increased cure rate was achieved by four weeks post-treatment. (FIG. 3) Glioma-bearing mice were treated with intratumoral interferon-gamma gene transfer, plus tumor lysate/CpG vaccine given s.c. on day 3, 7, and 14 post tumor. Luciferase in vivo tumor imaging showed 50% of the treated mice are tumor free one month later (signal near zero), whereas all the saline-treated mice (control) have large tumors.

### Example 5

### In Vivo Vaccination with Tumor Cell Lysate Plus CpG Oligodeoxynucleotides Eradicates Murine Glioblastoma

Glioblastoma Multiforme (GBM) is a lethal brain tumor that is a leading cause of solid tumor death in people under twenty, and accounts for 25% of all primary brain tumors in adults. Despite aggressive surgical resection and concurrent radiochemotherapy regimens, the prognosis for GBM patients remains extremely dismal with a two-year survival rate below 27%. The recent identification of brain tumor stem-like cells that are inherently resistant to radiation and chemotherapy, and are capable of tumor renewal, may partially account for the failures of current therapies. New treatments that are able to eradicate invasive and stem-like glioma cells are urgently needed. Immunotherapy has a theoretical appeal that tumor-reactive lymphocytes may infiltrate the brain parenchyma to “seek and destroy” tumor cells, including glioma stem-like cells, with greater precision than standard therapy.

A limiting obstacle to successful immunotherapy is the induction of adequate tumor antigen specific effector cells. To achieve this, tumor-associated antigens should be processed by antigen presenting cells (APCs) such as DCs, and the tumor antigens must be presented to T cells along with sufficient co-stimulatory signals to avoid tolerance. Based on this principle, various immunotherapy strategies have been employed for glioma, many of them using DCs pulsed with tumor lysate or tumor-associated peptides ex vivo. Several clinical trials have been conducted in which select glioma patients appeared to benefit from DC vaccines generated ex vivo. In these studies the induction of anti-tumor immune response was confirmed by DTH, ELISPOT, or HLA restricted tetramer staining. One constraint to these ex vivo vaccines is the requirement of purifying, culturing, and maturing DCs, which is not always possible and is an expensive process that requires significant expertise in DC manipulation. However, the direct in vivo administration of tumor-lysate with various adjuvants has not yielded satisfactory results, which was a motivating factor for developing the more complicated ex vivo vaccines.

Vaccination with irradiated glioma cells plus granulocyte monocyte-colony stimulating factor (GM-CSF) is capable of eliciting a curative immune response in highly immunogenic rat glioma models. However, this same vaccination method failed to cause regression in weakly immunogenic glioma models and has shown only modest clinical activity. Therefore, there has been intense interest in developing more potent approaches. Sandler et al. demonstrated that the combination of GM-CSF transduced, irradiated tumor cells plus CpG oligodeoxynucleotides (ODN) was a more potent immunotherapy than using GM-CSF transduced cells alone in an extracranial neuroblastoma model (Cancer Res 2003, 63:394-9). Nevertheless, recent attempts at scaling up GM-CSF transduced autologous glioma cell vaccines have met with significant technical hurdles, and it was concluded that simpler/alternative methods need to be developed.

TLR9 associates in the endosome with unmethylated CpG dinucleotide DNA sequences abundant in many bacteria, and potentiates a strong adaptive immune response to the invader. CpG ODNs are highly effective as vaccine adjuvants to directly stimulate the activation and maturation of DCs, thereby enhancing their ability to stimulate antigen-reactive T cells with strong anti-tumor activities in vitro and in vivo. Until now, CpG ODN has typically been administered intratumorally as single agent for the treatment of glioma. Despite curative effects observed in a rat glioma model, it was found that intratumoral CpG ODN administration was not effective against a weakly immunogenic GL261 mouse glioma model, which is consistent with preliminary clinical trial results. Initiating a therapeutic immune response by intratumoral therapy is challenging in glioma, because the tumor microenvironment is rich in immunosuppressive cytokines, and is heavily infiltrated by microglia with impaired antigen presenting capacity. It was hypothesized that in vivo administration of autologous tumor lysate plus CPG ODN could evoke an effective T cell-mediated response against glioma. Since this vaccine could be administered subcutaneously, it was also hypothesized it may overcome the immunosuppressive microenvironment of the glioma by priming T cells extracranially. The purpose of this study was to determine if effective antitumor immunity could be induced in vivo by subcutaneously administering CpG ODN mixed with tumor lysate (CpG/lysate) as vaccine in mice bearing intracranial glioma.

Materials and Methods

Cells and cell culture. GL261 is an aggressive glioma cell line that is derived from C57BL/6 mice and was obtained from Dr. P. Shrikant (Center for Immunology, Minneapolis, Minn.). C6 is a rat glioma cell line that was used an irrelevant control. The C6, parental/wild type (WT) GL261, and luciferase-stable GL261-Luc cells were maintained in DMEM supplemented with 10% FBS, 100 units/ml penicillin, 0.1 mg/ml streptomycin at 37° C., and 5% C02. GL261 and GL261-Luc tested negative for mycoplasma and murine parvo virus by PCR assay conducted routinely throughout the study.

Glioma model and in vivo imaging. Six to seven week old female C57BL/6 mice were purchased from Jackson Laboratory and maintained in a specific pathogen free (SPF) facility according to the guidelines of the University of Minnesota Animal Care and Use Committee (IACUC). For intracranial tumor inoculations, animals were deeply anesthetized with a ketamine/xylazine cocktail solution (53.7 mg/ml ketamine, 9.26 mg/ml xylazine) delivered at 1 ml/kg. 10,000 GL261-Luc cells in 1 μl of PBS were implanted stereotactically into the right striatum; coordinates were 2.2 mm lateral, and 0.5 mm posterior of bregma, and 3 mm ventral from the cortical surface of the brain. For imaging mice were deeply anesthetized by i.p. injection with avertin (225 mg/kg) and injected with 100 μl of luciferin (substrate for luciferase enzyme; 28.5 mg/ml, Xenogen®, Hopkinton, Mass.). Mice were imaged five minutes after luciferin injection using the Ivis 50 system (Xenogen®). A one-second grayscale exposure was overlayed with a five-minute luminescent exposure. Luciferase activity was analyzed using living image software (version 2.5; Xenogen®) according to the manufacturer's instructions.

CpG ODN and tumor cell lysate preparation. Purified CpG ODN 2006 (5′-TCGTCGTTTTGTCGTTTTGTCGTT-3′; SEQ ID NO:19) was obtained from Integrated DNA Technologies (Coralville, Iowa). CpG ODN 2006 was reconstituted in sterile pyrogen free water at a concentration of 20 μg/μl and stored at −80° C. for future use. To generate the cell lysate, 4×107 GL261 or GL261-Luc cells were collected and washed three times with PBS. Cells were then resuspended in 1 ml of PBS and lysed by five cycles of freezing at −80° C. and thawing at 37° C. in a water bath. Complete cell death was confirmed by using trypan blue exclusion. If any viable cells remained, freeze thaw cycles were repeated. The cell lysates were then stored at −80° C. until use.

Immunization protocol. Glioma-bearing mice were subcutaneously (s.c.) vaccinated on days 4, 11, and 18 after intracerebral inoculation. For each treatment 1 ml of tumor lysate was thawed and then 25 μl of a 20 μg/μl CpG solution was added and mixed. A 100 μl final volume containing 50 μg of CpG and lysate from 4×106 tumor cells was injected s.c. above the shoulders. All mice were anesthetized by intraperitoneal (i.p.) injection of ketamine/xylazine cocktail solution before the immunization. Control mice were injected identically with saline (100 μl), CpG (50 μg, 100 μl), or tumor cell lysate (lysate from 4×106 cells in 100 μl).

Lymphocyte depletion experiment. Specific lymphocyte populations were depleted in vivo by i.p. injection of 100 pg of anti-CD4 (clone GK1.5), anti-CD8 (clone 53-6.7) or anti-NK1.1 (clone PK136) antibodies (eBioscience, San Diego, Calif.) on days 1 and 2 before the first immunization. The mice were injected with the same antibodies one day before each additional immunization to maintain the depletion status. All the mice were immunized with CpG/lysate on days 4, 11, and 18 after tumor inoculation, except one group of mice that was treated with saline as the control (n=9-10/group).

Flow cytometry. Normal B6 mice were vaccinated two times, one week apart with CpG/lysate, CpG, lysate, or were not vaccinated as the controls (n=4/group). Six days after the last vaccination, the left and right cervical lymph nodes of each mouse were harvested, counted for LN cell numbers, and analyzed by flow cytometry with various monoclonal antibodies (1 μg/106 cells) including FITC-anti-CD3, PE-anti-CD8, APC-anti-CD4, FITC-anti-CD1 1c, PE-anti-CCR7, and APC-anti-CD86 (eBioscience, San Diego, Calif.).

Elispot and CTL assays. To evaluate tumor-reactive lymphocytes, glioma-bearing mice were vaccinated with CpG/lysate, lysate alone, CpG alone, or saline on days 4 and 7 after tumor inoculation. Five days after the second immunization, splenocytes were isolated using ficoll gradient centrifugation and evaluated using an IFN-gamma ELISPOT kit (Cell Sciences®, Canton, Mass.) according to the manufacturer's instructions. Briefly, splenocytes were distributed in 96 well PVDF plates coated with mouse IFN-gamma capture antibody at a concentration of 5×105/well. 5×104 mitomycin C-treated GL261-Luc cells or GL261-WT cells were used as stimulus. After incubation for 48 hrs at 37° C. in a 5% CO2 incubator, plates were washed and incubated with biotinylated detection antibody for 1.5 hrs at room temperature (RT). A streptavidin alkaline phosphatase detection solution was then incubated for 1 hr at RT, followed by a final washing step. Spots were developed by adding substrate (BCIP/NBT) buffer. The plates were washed, dried, and read using an ELISPOT reader (CTL Immunospot®, Cleveland, Ohio).

The LDH (lactate dehydrogenase) release method (28, 29) was used for the CTL assay. Glioma-bearing mice were vaccinated identically to the ELISPOT experiment. Seven days after the last immunization, splenocytes were harvested and (5×06/well) were stimulated with mitomycin C-treated GL261-Luc cells at a ratio of 20:1 in 24 well plates in 2 ml of culture medium. Next, 10 IU/ml of IL-2 was added at day two and day four. After six days of culture, lymphocytes were collected and co-cultured with target cells, GL261-Luc (1×104/well) or GL261-WT (1×104/well), in 96 well plates in triplicate at various effector cell/target cell (E/T) ratios (100:1, 50:1, 25:1, 5:1, 1:1).

After 4 hrs, a 100-μl reaction mixture was added and incubated for 10 minutes at RT. The reaction was stopped by 50 μl of stop solution, and the plate was read at 490 nm. Spontaneous lysis was measured from wells containing only target cells or various numbers of effector cells. To determine cell-mediated cytotoxicity, background values were subtracted from each sample, the absorbance of the triplicate samples were averaged, and specific lysis was calculated according to the following formula: Cytotoxicity (%)=(effector/target cell mix−effector cell control)−low control/high control−low control.

Statistical analysis. Statistical comparisons were done using a one-way ANOVA, and ad hoc comparisons using two-tailed student's t-test with Prism 4 software (Graph Pad Software, Inc., San Diego, Calif.); P values <0.05 were considered significant. Differences in animal survival between treatment groups were evaluated by log-rank statistical analysis with Sigmastat software (Systat Software, Inc. San Jose, Calif.); only P values <0.05 were considered significant.

Results

CpG/lysate vaccination increases the number of T cells and activated DCs in the draining lymph nodes.

The ability of CpG ODN to activate and mature DCs to elicit T cell-mediated responses has been established. An experiment was conducted to characterize the effects of CpG/lysate vaccination on DCs and T cells in the draining lymph nodes. Normal B6 mice were vaccinated two times, one week apart, by subcutaneous (s.c.) injection with CpG ODN, tumor lysate, or CpG/lysate (n=4/group). Six days after the second vaccination, the draining cervical lymph nodes of each mouse were harvested, counted for total cell number, and analyzed by flow cytometry.

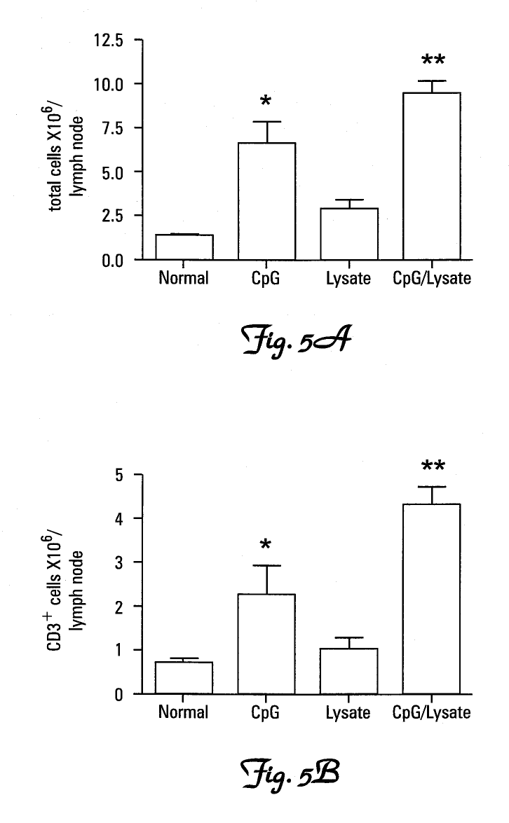

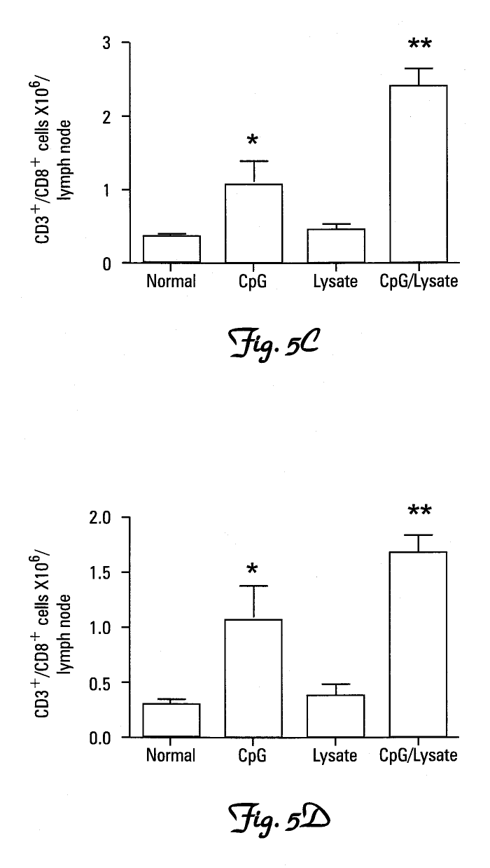

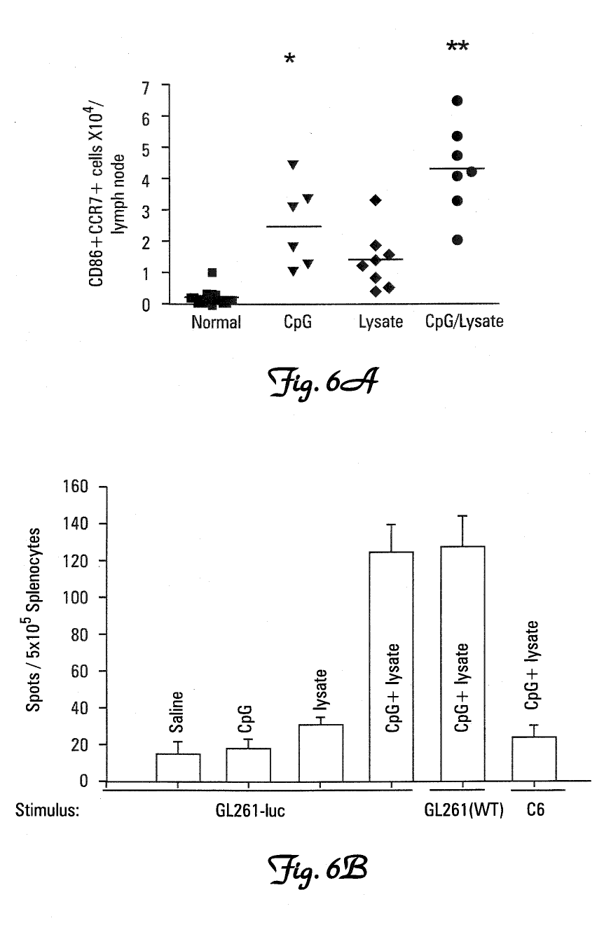

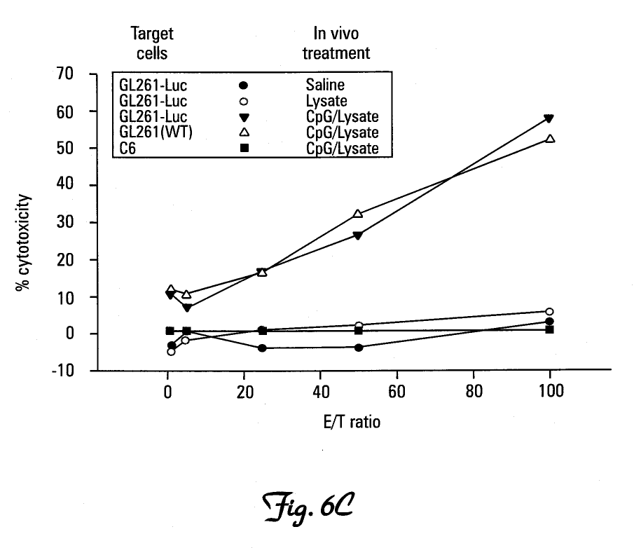

There was a significant expansion in the number of total lymph node cells (FIG. 5A) in CpG/lysate vaccinated mice, with the cell counts higher than that of mice injected with CpG ODN or tumor lysate, respectively. This was further characterized as an expansion of CD4+ and CD8+ T cells in the lymph nodes of CpG/lysate-treated mice compared to all groups (p<0.05; FIG. 5B-D). In addition, draining lymph nodes from CpG ODN and CpG/lysate-treated groups both had a significant increase in the number of activated (CD11c+CD86+/CCR7+) DCs, with the CpG/lysate group having a higher accumulation of CD1 1c+CD86+/CCR7+DCs than that of CpG ODN group (p=0.039; FIG. 6A).

CpG/lysate vaccination induces the generation of tumor-reactive lymphocytes.

To determine if tumor-reactive lymphocytes were generated in response to CpG/lysate vaccination, groups of glioma-bearing mice were vaccinated with CpG ODN, tumor lysate, CpG/lysate, or saline. Splenocytes were harvested five days after the last vaccination and co cultured with GL261 or GL261-Luc cells to measure IFN-gamma elaboration by the ELISPOT assay. Splenocytes from mice vaccinated with CpG/GL261-Luc lysate exhibited a six-fold increase in IFN gamma spots compared to all controls (p<0.001; FIG. 6C). There was no significant difference found when splenocytes were stimulated with GL261 or GL261-Luc cells, suggesting luciferase was an irrelevant antigen. Very similar trends were observed when the CTL assay was conducted to measure the ability of effectors to lyse GL261 or GL261-Luc in vitro. Only splenocytes harvested from mice vaccinated with CpG/lysate had appreciable activity against GL261 and GL261-Luc compared to all control groups (FIG. 6D). Taken together, these results demonstrated that s.c. vaccination with glioma cell lysate plus CpG ODN generates tumor-reactive lymphocytes capable of killing glioma cells from which the lysate was derived.

CpG/lysate vaccination can effectively eradicate or significantly reduce the growth of glioblastoma in mice.

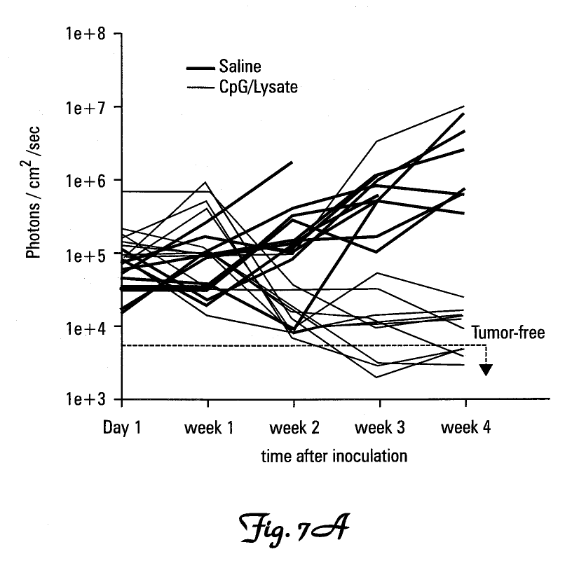

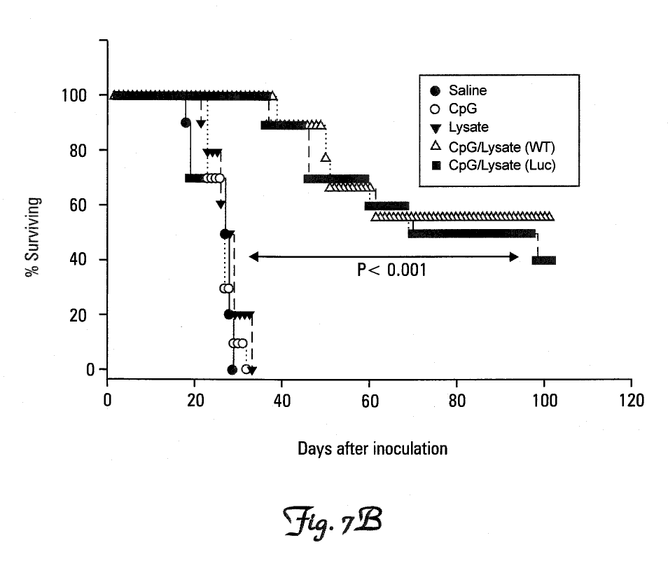

In order to determine the efficacy of CpG/lysate vaccination against glioma, mice were intracerebrally inoculated with GL261-Luc cells. Glioma-bearing mice were vaccinated with CpG ODN, tumor lysate, CpG/lysate, or saline on days 4, 11, and 18 after tumor inoculation. An additional cohort of mice was vaccinated with lysate derived from the parental “WT” GL261 plus CpG to investigate if the expression of luciferase in GL261-Luc would bias the response to vaccination (n=9-10/group). Bioluminescent imaging revealed that mice vaccinated with CpG/lysate exhibited delayed tumor growth or complete tumor regression (FIG. 7A). Treatment with CpG, lysate, or saline alone had no effect on tumor growth or survival, with all mice dying within 32 days (FIG. 7A). CpG/lysate-treated mice survived significantly longer than controls (p<0.001), with a median survival beyond 80 days compared to 27-29 days in all control groups (FIG. 7B). All mice that experienced complete tumor regression measured by imaging survived beyond 100 days. Two of five mice that were treated with CpG/GL261-luc lysate had detectable tumor when imaged at day 70. One of these animals eventually died at day 92. Five of nine animals treated with CpG/GL261 lysate had no measurable tumor at day 70 and survived beyond 100 days (FIG. 7B). There was not a statistically significant difference in the survival of mice vaccinated with either CpG/GL261 lysate or CpG/GL261-Luc lysate, which indicated luciferase did not cause or influence the regression of GL261-Luc tumors.

CD4+ T lymphocytes play a pivotal role in CpG/Lysate vaccine-induced tumor eradication

To determine the role of specific subsets of lymphocytes has in CpG/lysate vaccine-induced tumor eradication, we conducted a depletion experiment by administering anti-CD4, anti-CD8, or anti-NK1.1 antibody prior to each vaccination of glioma bearing mice with CpG/lysate. Several mice were sacrificed after antibody injection. Flow cytometry analysis on splenocytes confirmed greater than 98% depletion of the indicated cell population. Bioluminescent imaging showed that CD4 depletion completely abolished the tumor inhibitory effect of CpG/lysate vaccination in the majority of mice. In addition, CD8 or NK cell depletion also significantly diminished the tumor inhibitory effect of CpG/lysate vaccination. Consistent with the imaging data, mice that were depleted of CD4 cells did not survive significantly longer than mice treated with saline and no depletion. Mice that were depleted of CD8 or NK cells did survive significantly longer than saline controls (p<0.001), but none survived beyond 45 days. In contrast, 60% of mice that were vaccinated without any depletion survived more than 60 days, a difference that was significant compared to CD8-depleted or NK-depleted mice (P<0.001). These results reveal that CD4+ lymphocytes played a pivotal role in tumor rejection in response to CpG/lysate vaccination, and that CD8+ and NK cells also contributed significantly to CpG/lysate vaccine-induced anti-tumor immunity.

Discussion

Vaccination with DCs pulsed with antigen ex vivo has been employed and demonstrated anti-tumor activity in a fraction of glioma patients. This approach requires significant expertise and costs to culture DCs used in the personalized vaccine. The attempts at clinical use with autologous GM-CSF transduced glioma cell vaccines have also met with limited success and significant technical hurdles, again highlighting the need to develop alternative approaches. The development of a cell-free cancer vaccine system may streamline a cost effective and clinically feasible protocol, and consequently allow more patients to be treated. It has been shown that TLR9 stimulation with CpG ODN increases the effectiveness of lysate-pulsed DC vaccines prepared ex vivo in experimental cancer models. CpG ODN potently enhances DC activation, maturation, survival, and thereby promotes a more robust adaptive anti-tumor immune response. The results of the current study demonstrate that direct in vivo administration of tumor cell lysate along with CpG ODN has potent anti-tumor efficacy in this mouse glioma model, achieving a cure rate of up to 55%. This cure rate is comparable to previous studies that utilized ex vivo lysate pulsed DC vaccines against GL261 glioma (40-80% cure). The combined vaccination with CpG/lysate resulted in a significant increase in activated DCs in the draining lymph nodes, and a significant expansion of T cells that elaborated IFN-gamma and lysed the glioma cells from which the lysate was derived. The data support the hypothesis that DCs engulf CpG ODN along with the tumor lysate and traffic to lymphoid organs to present tumor-associated antigens to T cells. The addition of CpG to the tumor lysate was absolutely required to induce any of the abovementioned effects. Consistent with this, only animals treated with CpG/lysate exhibited delayed tumor growth or prolonged survival compared to all other groups. These results support our second hypothesis that priming T cells extracranially with CpG-activated APCs pulsed with tumor antigens is superior to direct intratumoral CpG ODN administration, since the later failed to cause sustained tumor regression in the identical GL261-Luc model. It has been shown CpG ODN can exert significant toxicity when administered directly into the CNS, including causing seizures or meningitis. Taken together, the results of the current study reveal a more effective, simple, and potentially safer method in the administration of CpG ODN for glioma immunotherapy.

Mechanistic studies revealed that both CD4+ and CD8+ cells played an important role in tumor regression. This phenomenon has been reported by previously using CpG immunotherapy in a murine neuroblastoma model, and is possibly due to CD4-mediated elaboration of IL-2 and other cytokines that promote a strong CTL response against tumor. Similarly, the importance of NK cells and NK1.1+/CD 11c+“killer” DCs in contributing to CpG-induced anti-tumor immunity has been documented previously. It is likely that macrophages also played a role in the anti-tumor immune response, because nearly all subsets of APCs in mice express TLR9, whereas in humans the expression of TLR9 may be restricted to plasmacytoid DCs and B cells. Since these APCs are recruited to sites of inflammation, it is plausible this CpG/lysate vaccine strategy could translate into humans. Despite the restricted expression of human TLR9, the “type B” CpG ODN used in these studies has been shown to modulate cytokine production and/or proliferation of human B cells, monocytes, plasmacytoid DCs, and NK cells via direct or indirect mechanisms. An additional consideration is that human keratinocytes in the vaccination site express TLR9 and secrete a variety of inflammatory cytokines and chemokines upon CpG ODN exposure. Accordingly, CpG ODNs have shown anti-tumor activity in patients with lung cancer and melanoma.

The CpG ODN used in this study (CpG 2006 or CpG 7909) is optimal for activating human TLR9, but can also cross-react with mouse TLR9. CpG 2006 was used rather than a more potent mouse-specific CpG ODN in order to put this immunotherapy to a more stringent test in vivo, circumvent overestimating the potency in the murine model, and ensure more rapid applicability to human use. A dosage of about 50 μg is proposed. The dose of 50 μg equates to approximately 2.5 mg/kg in an adult mouse weighing 20 grams. This dose is modestly higher than what has been administered in melanoma patients by subcutaneous injection, which was up to 0.8 mg/kg.

### Example 6

The Examples above describe a cancer vaccine for the treatment of glioma brain tumors. The method of vaccination was to mix a tumor cell lysate with immunostimulatory toll-like receptor (TLR) agonists and inject this beneath the skin. This method is applicable to other types of cancers as well.

Similar to the original ex vivo tumor lysate/CpG vaccine for glioma, this same vaccine worked to treat breast cancer in mice. Mice bearing breast cancer were cured or tumor growth was delayed when vaccinated with tumor cell lysate derived from their tumor and a TLR9 agonist (CpG). Thus, this method may be used for any intracranial or extracranial tumor including breast cancer and lung cancer.

The vaccine has been modified for breast cancer entirely in vivo (no ex vivo work required). By intratumorally injecting a breast tumor in mice with an agent that causes tumor lysis (diphtheria toxin) along with CpG (TLR9 agonist), a tumor lysate/CpG vaccine was generated in situ to treat these tumors. This is different from the Examples above in which the lysate is made outside of the body by multiple freeze-thaw cycles that break open the tumor cells, and then injected back into the body with CpG. In this case, the entire vaccine is made in the living animal or patient. Thus, in an alternative method, one directly co-injects a cytotoxic agent along with TLR agonists to generate a tumor cell lysate/TLR agonist vaccine in situ. The cytotoxic agent could be a chemotherapy drug or biological toxin.

In other embodiments, any residual primary breast tumors are surgically resected after vaccination is complete, such that metastatic cancer is abolished, and the subject is protected from intracranial tumor engraftment. This approach can be applied to patients before surgery, to prevent tumor recurrence at distal sites in the body.

1. A pharmaceutical composition comprising a toll-like receptor (TLR) agonist and a tumor lysate and/or tumor lysis agent in a pharmaceutically acceptable carrier.

2. The pharmaceutical composition of claim 1, wherein the TLR agonist is a TLR-9 agonist or a TLR-3 agonist.

3. The pharmaceutical composition of claim 2, wherein the TLR agonist is an oligonucleotide of 8-1000 bases in length containing an immunostimulatory CpG motif.

4. The pharmaceutical composition of claim 3, wherein the oligonucleotide has a natural phosphodiester backbone, a completely or partially synthetic backbone, or a synthetic phosphorothioate backbone.

5. The pharmaceutical composition of claim 3, wherein the oligonucleotide is made with a chimeric backbone with synthetic phosphorothioate linkages at the 3′ and 5′ ends and natural phosphodiester linkages in the CpG-containing center to form a chimeric oligonucleotide.

6. The pharmaceutical composition of claim 5, wherein the chimeric oligonucleotide is made with synthetic phosphorothioate linkages for five linkages at the 3′ end and two linkages at the 5′ end, and with natural phosphodiester linkages in between.

7. The pharmaceutical composition of claim 3, wherein the oligonucleotide has a formula 5′-N₁X₁CGX₂N₂-3′, wherein at least one nucleotide separates consecutive CpGs; X₁ is adenine, guanine or thymidine; X₂ is cytosine, adenine, or thymine; N₁ is a nucleic acid of about 0-26 bases; N₂ is a nucleic acid of about 0-26 bases.

8. The pharmaceutical composition of claim 7, wherein neither N₁ nor N₂ contains a CCGG quadmer or more than one CGG trimer and wherein the oligonucleotide is from about 8-30 bases in length.

9. The pharmaceutical composition of claim 3, wherein the oligonucleotide has a formula: 5′-N₁X₁X₂CGX₃X₄N₂-3′, wherein at least one nucleotide separates consecutive CpGs; X₁X₂ is selected from the group consisting of TpT, CpT, TpC, ApT, GpT, GpG, GpA, and ApA; X₃X₄ is selected from the group consisting of GpT, GpA, ApA, ApT, TpT and CpT; N₁ is a nucleic acid of about 0-26 bases, and N₂ is a nucleic acid of about 0-26 bases.

10. The pharmaceutical composition of claim 9, wherein N₁ and N₂ do not contain a CCGG quadmer or more than one CGG trimer; and the oligonucleotide is from about 8-1000 bases in length.

11. The pharmaceutical composition of claim 9, wherein the oligonucleotide is from about 8-30 bases in length.

12. The pharmaceutical composition of claim 2, wherein the TLR-3 agonist is poly-ICLC.

13. The pharmaceutical composition of claim 1, further comprising at least one adjuvant.

14. The pharmaceutical composition of claim 13, wherein the at least one adjuvant contains aluminum (alum).

15. The pharmaceutical composition of claim 14 wherein the aluminum-containing adjuvant is aluminum hydroxide.

16. The pharmaceutical composition of claim 1, further comprising interferon-gamma or a vector encoding interferon-gamma.

17. The pharmaceutical composition of claim 1, wherein the TLR agonist comprises more than one type of oligonucleotide.

18. The pharmaceutical composition of claim 1, wherein the tumor lysis agent is a chemotherapy drug or biological toxin.

19. The pharmaceutical composition of claim 1, wherein the tumor lysis agent is diphtheria toxin.

20. The pharmaceutical composition of claim 1, wherein the tumor lysis agent is temozolomide (Temodar®), Temodar, Carboplatin, Doxyrubicin, or a replication competent CMV virus.

21. A method of inducing a therapeutic immune response in a subject having or at risk of having a tumor, comprising administering to the subject a therapeutically effective amount of the pharmaceutical composition of claim 1.

22. The method of claim 21, wherein the subject is a mammal.

23. The method of claim 21, wherein the subject is a human.

24. The method of claim 21, wherein the tumor lysate comprises lysed tumor cells from the subject.

25. The method of claim 21, wherein the tumor lysate comprises lysed tumor cells from an allogenic cell line.

26. The method of claim 21, wherein the TLR agonist and the tumor lysate or the tumor lysis agent are administered simultaneously.

27. The method of claim 21, wherein the tumor lysate or the tumor lysis agent and the TLR agonist are mixed ex vivo.

28. The method of claim 21, wherein the tumor lysate or the tumor lysis agent and the TLR agonist are administered separately within 21 days of each other.

29. The method of claim 28, wherein the wherein the tumor lysate or the tumor lysis agent and the TLR agonist are administered separately within 2-5 days of each other.

30. The method of claim 21, wherein the tumor lysate or the tumor lysis agent and the TLR agonist are administered multiple times.

31. The method of claim 30, wherein the tumor lysate or the tumor lysis agent and the TLR agonist are administered 2-5 times.

32. The method of claim 21, wherein the pharmaceutical composition is administered intratumorally.

33. The method of claim 21, further comprising administering gamma-interferon or a vector encoding interferon-gamma.

34. The method of claim 21, wherein the tumor is a glioma brain tumor, a breast tumor or a lung tumor.

35. A method of inducing an immune response in a subject, comprising administering to the subject a therapeutically effective amount of the pharmaceutical composition of claim 1.

36. A method of preventing metastatic spread of a tumor in a subject having received a primary therapy comprising administering the pharmaceutical composition of claim 1.
